(19) United States
(12) Patent Application Publication
(10) Pub. No.: US 2003/0190329 A1
(43) Pub. Date: Oct. 9, 2003

(54) Coxsackievirus vectors and their use in prevention and treatment of disease

(76) Inventors: Steven M. Tracy, Omaha, NE (US); Nora M. Chapman, Omaha, NE (US)

Correspondence Address: Janet E. Reed, Esq., Woodcock Washburn LLP, 46th Floor, One Liberty Place, Philadelphia, PA, 19103, US

(21) Appl. No.: 09/817,748

(22) Filed: Mar. 26, 2001

Related U.S. Application Data
(63) Continuation-in-part of application No. 09/403,672, filed on Mar. 27, 2000, now Pat. No. 6,323,024.
Application No. 09/403,672 is a 371 of international application No. PCT/US98/04291, filed on Mar. 5, 1998.

(51) Int. Cl. A61K 39/21; A61K 39/125; C12N 15/867

(52) U.S. Cl. 424/208.1; 424/216.1

(57) ABSTRACT

The present invention is drawn to the use of attenuated coxsackievirus cardiotropic virus vectors as efficient gene transfer vectors to deliver immunomodulatory or other biologically active proteins and/or antigenic epitopes in transient infections to aid in preventing, ameliorating, and/or ablating infectious viral heart disease and reducing, or ablating entirely, heart transplant rejection. Specifically disclosed are univalent and multivalent vaccines for certain viruses, including adenovirus and coxsackieviruses. Also disclosed are compositions and methods for suppressing onset of type 1 diabetes, using vectors of the invention that express immunomodulatory proteins, specifically IL-4.

This application is a continuation-in-part of U.S. application Ser. No. 09/403,672, having a filing date of Mar. 27, 2000 and claiming priority under 35 U.S.C. §371 to International Application No. PCT/US98/04291, which itself claims priority under 35 U.S.C. §120 to U.S. application Ser. No. 08/812,121, filed Mar. 5, 1997, now U.S. Pat. No. 6,071,742, issued Jun. 6, 2000. The entireties of each of the above-listed applications are incorporated by reference herein.

The United States government has certain rights in the invention described herein, which was made in part with funds from the National Institutes of Health.

### FIELD OF THE INVENTION

The present invention relates generally to the fields of molecular biology and virology. More specifically, the present invention relates to an attenuated Coxsackievirus, its use as a delivery vehicle for nucleic acids encoding antigenic or biologically active proteins, and treatment or prevention of viral infection or type 1 diabetes.

### BACKGROUND OF THE INVENTION

Various scientific articles, scholarly publications and patent documents are referred to herein to describe the state of the art to which the invention pertains. Each of these documents is incorporated by reference herein in its entirety.

The coxsackieviruses, members of the family Picornaviridae, are divided into two groups, based essentially on their pathogenicity and replication in newborn mice. The Group B coxsackieviruses (CVB) are composed of six serotypes (1-6). Coxsackievirus capsids are 29-nm-diameter icosahedral structures with the typical enterovirus canyon-like depressions surrounding the fivefold axes, which, by analogy to polioviruses and rhinoviruses, are binding sites for the cell membrane receptor human coxsackievirus adenovirus (Ad) receptor (HCAR).

Similar to other members of the Picornaviridae, the CVB genome is a single-stranded, messenger sense, polyadenylated RNA molecule (for review see Romero, J. R. et al., Current Topics in Microbiology and Immunology 223: 97-152, 1997). Genome analysis of the CVB shows that they are organized into a 5′ nontranslating region, a protein coding region containing a single open reading frame, a 3′ nontranslated region and a terminal poly-A tail, similar to other Picornaviruses. The CVB protein coding region can be further divided into three regions, P1, P2 and P3. P1 encodes the four capsid proteins VP4 (1A), VP2 (1B), VP3 (1C) and VP 1 (1D); P2 and P3 encode the non-structural proteins required for the CVB lifecycle: 2A (protease), 2B, 2C, 3A 3B (Vpg), 3C (protease) and 3D (polymerase) (See Romero et al., 1997, supra).

The genomes of CVB that have been fully sequenced are very similar to one another in length, ranging from 7389 nucleotides (CVB1) to 7402 nucleotides (CVB5) (Romero et al., 1997 supra). Variations in length are due to differences within the coding region of VP1 and VP2 (capsid proteins) and in the 5′ and 3′ non-translated regions. The 5′ non-translated regions also show remarkable similarity in length. For a detailed review of the similarities among the CVB genomes, refer to Romero et al, supra, 1997.

One of the six serotypes of the group B coxsackieviruses, Coxsackievirus B3 (CVB3), has been particularly well studied, and serves as a prototype for the other coxsackieviruses. The CVB3 genome is single molecule of positive sense RNA which encodes a 2,185 amino acid polyprotein. The single long open reading frame is flanked by a 5′ non-translated region (5′ NTR), 742 nucleotides long, and a much shorter 3′ NTR which terminates in a polyadenylate tract. Like the polioviruses (PVs), CVB3 shuts off host cell protein translation in infected HeLa cells. The near atomic structure of the CVB3 virion has been solved, demonstrating that the CVB3 capsid shares a similar capsid structure with genetically-related entero-and rhinoviruses.

Coxsackie B viruses are established etiologic agents of acute human inflammatory heart disease (reviewed in Cherry, J. D. Infectious Diseases of the Fetus and Newborn Infant, 4^{th} ed., pp.404-446, 1995) and cardiac CVB3 infections may lead to dilated cardiomyopathy. Systemic CVB3 infections are common in neonates: often severe or life-threatening, they usually involve inflammation and necrosis of the heart muscle. One study of neonates under three months of age suggested a CVB infection rate as high as 360/100,000 infants with an associated 8% mortality (Kaplan, M. H., et al., Rev. Infect. Dis. 5:1019-1032, 1983). Acute and chronic inflammatory heart disease afflicts approximately 5-8 individuals per one hundred thousand population annually worldwide (Manolio, T. A., et al. Am. J. Cardiol. 69: 1458-1466, 1992). Based upon molecular evidence of enteroviral involvement, approximately 20-30% of cases of acute inflammatory heart muscle disease and dilated cardiomyopathy involve an enteroviral etiology (see, e.g., Kandolf, R. Coxsackieviruses—A General Update, p. 292-318, 1988; and Martino, T. A., et al., Circ. Res. 74:182-188, 1994), and murine models of experimental CVB-induced myocarditis exist that recapitulate many aspects of the human disease counterpart. More recently, human Ad DNA has been detected in hearts of patients with myocarditis (Martin, A. B. et al., Circulation 90: 330-339, 1994), with subsequent sequence analysis of the amplimers from diseased hearts shown to be consistent with infections by Ad2 (Pauschinger, M. N. et al., Circulation 99: 1348-1354, 1999). There are no commercially available vaccines against either CVB or Adenovirus.

The inflammatory process which characterizes enterovirus-induced inflammatory heart disease has been extensively studied in murine models (reviewed in Gauntt, C., et al., Medical Virology, 8th ed., p. 161-182, 1989; Leslie, K., et al., Clin. Microbiol. Rev. 2:191-203, 1989; Sole, M., and P. Liu., J. Amer. Coll. Cardiol. 22 (Suppl.A):99A-105A, 1994; and Woodruff, J. F., Am. J. Pathol. 101:425-484, 1980), but it remains unclear precisely what specific roles are played by the various components of the cell-mediated immune response in the induction of acute disease and continuation of the chronic state. However, it is clear that in the presence of an intact murine immune system, CVB3-induced inflammatory heart disease develops only following inoculation of mice with a cardiovirulent CVB3 strain (Chapman, N. M., et al., Arch. Virol. 135:115-130, 1994; Gauntt, C. J., et al., J. Med. Virol. 3:207-220, 1979; Tracy, S., et al., Arch. Virol. 122:399-409, 1992; and Woodruff, J. F., and E. D. Kilbourne, J. Infect. Dis. 121:137-163, 1970).

Both cardiovirulent (able to induce disease) and non-cardiovirulent strains of CVB3 replicate well in hearts of experimentally-infected mice. Only cardiovirulent CVB3 strains, however, cause the significant cardiomyocyte destruction with subsequent cardiac inflammation which is characteristic of acute myocarditis (Chapman, N. M., et al., Arch. Virol. 135: 115-130 (1994); and Tracy, S., et al., Arch. Virol. 122:399-409, 1992). Non-cardiovirulent CVB3 is cleared from the experimentally-infected murine heart within 7-10 days post-infection, while infectious cardiovirulent CVB3 can remain detectable in hearts for up to 2 weeks post-infection (Klingel, K., et al, Proc. Natl. Acad. Sci. U.S.A. 89:314-318, 1992; Lodge, P. A., et al., Am. J. Pathol. 128:455-463, 1987; and Tracy, S., et al., Arch. Virol. 122:399-409, 1992). The fall in murine cardiac infectious CVB3 titer is coincident with the rise in anti-CVB3 neutralizing antibody titers and the ability of T cells to recognize CVB3 antigens (Beck, M. A., and S. Tracy, J. Virol. 63:4148-4156, 1989; Gauntt, C., et al., Medical Virology, 8th ed., p. 161-182, 1989; and Leslie, K., et al., Clin. Microbiol. Rev. 2:191-203, 1989). In addition to direct in situ hybridization evidence for enteroviral replication in human heart myocytes and for cardiovirulent CVB3 replication in murine heart myocytes, CVB3 infects a variety of cultured cardiac cell types including murine and human cardiomyocytes, murine fetal heart fibroblasts and cardiac endothelial cells.

Of great interest is that heart transplantation and acute enteroviral heart disease evoke a similar immune response in a host. Acute rejection of a transplanted heart can involve primarily a Th1 type T cell response, the same type of T cell response that is observed in CVB3 induction of acute myocarditis in well-studied murine models of CVB3-induced inflammatory heart disease. Switching of this response to the Th2 type response, with a concomitant ablation of disease, has been accomplished in mice through parenteral administration of the key modulatory cytokines IL-4 or IL-10. However, parenteral administration of cytokines to humans often results in undesired clinical side effects.

Thus, the prior art is deficient in the use of an attenuated coxsackievirus as a gene delivery vector, specifically to target immunomodulatory or other biologically active genes or antigenic epitopes to selected cells, tissues or organs, including the heart. Such a mode of administration or gene delivery circumvents the undesirable side effects of parenteral administration of immunomodulatory agents, antigens or other therapeutic molecules. Thus, the present invention fulfills this long-standing need and desire in the art.

### SUMMARY OF THE INVENTION

The present invention provides viral vectors for therapeutic or prophylactic use in human disease by delivering nucleic acids encoding antigenic epitopes or specific biologically active gene products, such as (but not limited to) immunomodulatory cytokines, to target cells, tissues or organs in an individual.

Thus, according to one aspect of the invention, a viral vector for delivering a heterologous nucleic acid to a target cell, tissue or organ is provided, which comprises a coxsackievirus genome modified to encode an attenuated coxsackievirus, the genome further comprising at least one cloning site for insertion of at least one expressible heterologous nucleic acid. In a preferred embodiment, the coxsackievirus genome is a coxsackievirus B genome, most preferably a coxsackievirus B3 genome.

In one embodiment of the invention, attenuation of the coxsackievirus is achieved by altering a transcription regulatory region of the genome. Preferably, the transcription regulatory region comprises a 5′ untranslated region of the genome. In one embodiment, the 5′ untranslated region is replaced with a 5′ untranslated region of a non-coxsackievirus enterovirus genome selected from the group consisting of poliovirus and echovirus. In another embodiment, a coxsackievirus B3 genome is modified by substituting a C or G for a U at nucleotide position 234 of the genome.

The cloning site of the coxsackievirus vector can be positioned between a coding sequence for a capsid protein and a coding sequence for viral protease. In another embodiment, the cloning site is positioned at the start of the genome's open reading frame, and is constructed such that the inserted expressible heterologous DNA comprises a translation start codon and a 3′ sequence recognized by a viral protease.

In one embodiment, the expressible heterologous DNA carried by the coxsackievirus vector of the invention encodes an antigenic product. In another embodiment, it encodes a biologically active product, such as a biologically active protein. Preferably, the protein is a cytokine, such as IL-4 or IL-10. Alternatively, the protein could be another immunomodulatory protein, such as B-7 (B-7-1 or B-7-2).

According to another aspect of the present invention, there is provided a bioengineered virus for the therapeutic delivery of at least one heterologous gene to a target organ or organ system in an individual, comprising a Coxsackievirus B3 (CVB3), wherein said Coxsackievirus B3 is attenuated, and wherein a genome of said CVB3 codes for said at least one heterologous gene. Attenuation of the CVB3 may be accomplished through a transcriptional mechanism. Preferred embodiments include attenuating the virus by substituting a cytosine or guanosine nucleotide for a uracil nucleotide at position nt234 in the genome of the coxsackievirus B3. Another preferred embodiment includes point mutations at positions nt233 and nt236 in the genome of the Coxsackievirus B3, or deletion entirely of nt 233-236.

In addition, the 5′ non-translated region of the genome of the Coxsackievirus B3 may be substituted with a 5′ non-translated region of a genome from a non-enterovirus to achieve attenuation. In a preferred embodiment, the non-enterovirus is a poliovirus or echovirus.

In most preferred embodiments, the genome of the bioengineered Coxsackievirus B3 includes the basic CVB3/0 genome (as reported by Chapman, N. M., et al, Arch. Virol. 135: 115-130 (1994)), wherein a coding sequence for a heterologous gene is inserted between a capsid protein coding sequence and a viral protease coding region site. Alternatively, a heterologous gene may be inserted at the start of the open reading frame, directly upstream of capsid protein 1A, start with the initiation codon AUG, and end with a sequence recognized by a viral protease. In this preferred embodiment, an immunomodulatory gene or a gene for an antigenic epitope is used. In a more preferred embodiment, cytokine genes are delivered. In a most preferred embodiment, the cytokine is IL-4 or IL-10. Up to seven cytokine genes may be delivered in one vector. Further, both antigenic epitopes and cytokines may be delivered at the same time. Also, a preferred embodiment utilizes sequences for viral proteases P2-A and P3-C.

According to another aspect of the present invention, a method is provided for suppressing an immune response in an individual, comprising the step of administering the bioengineered therapeutic virus containing an immunomodulatory gene to an individual.

According to another aspect of the present invention, a method is provided for vaccinating an individual, comprising the step of administering the bioengineered therapeutic virus containing a gene for an antigenic epitope to an individual.

Specific vaccines and vectors encoding biologically active molecules are also provided in accordance with the present invention, along with method for their use.

Thus, a preferred embodiment of the invention provides a vaccine for immunizing an individual against a virus, specifically adenovirus, HIV or various coxsackieviruses, wherein the vaccine is a viral vector comprising a coxsackievirus genome modified to encode an attenuated coxsackievirus, the genome further comprising at least one cloning site for insertion of at least one expressible heterologous nucleic acid, wherein the heterologous nucleic acid encodes at least one antigenic epitope of the virus. In a preferred embodiment, the virus is adenovirus and the heterologous nucleic acid encodes an Adenovirus 2 hexon loop. In another embodiment, the virus is human immunodeficiency virus. In another embodiment, the vaccine is adapted to immunize an individual against a plurality of viruses. As one example, the plurality of viruses comprise a plurality of coxsackievirus serotypes and the heterologous nucleic acid encodes a BC loop of capsid protein 1D from one or more coxsackievirus serotypes other than the viral vector serotype.

According to another aspect of the invention, a composition for treating an individual for insulin-dependent diabetes mellitus is provided. The composition features a viral vector comprising a coxsackievirus genome modified to encode an attenuated coxsackievirus, the genome further comprising at least one cloning site for insertion of at least one expressible heterologous nucleic acid, wherein the heterologous nucleic acid encodes a biologically active immunomodulatory protein that induces a shift from a Th1 to a Th2 immune response in the individual. In a preferred embodiment, the heterologous nucleic acid encodes IL-4.

According to another aspect of the invention, a method is provided for treating, preventing or suppressing onset of insulin-dependent diabetes mellitus in an individual. The method comprises administering to the individual the aforementioned viral vector that expresses IL-4 or another suitable immunomodulatory protein.

According to another aspect of the invention, method is provided for suppressing onset of insulin-dependent diabetes mellitus in an individual. This method comprises inoculating the individual as a juvenile or infant with a coxsackievirus, preferably a CVB3, and most preferably a virulent strain of CVB3. The inventors have discovered that inoculation of individuals with these viruses at an early age effectively suppresses the onset of insulin-dependent diabetes mellitus as an individual ages.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

The appended drawings have been included herein so that the above-recited features, advantages and objects of the invention will become clear and can be understood in detail. These drawings form a part of the specification. The appended drawings illustrate preferred embodiments of the invention and should not be considered to limit the scope of the invention.

FIG. 1 shows the mIL4 insert in the CVB3/0-IL4 genome. The mIL4 sequence has been cloned between the viral capsid protein P1-D and the viral protease 2A (P2-A). During translation of the viral polyprotein, the most likely mechanism is that the protease P2-A cleaves itself out of the nascent protein in cis and cleaves the site between the capsid protein P1-D and mIL4 sequence in trans. Nucleic acid sequence on top line is SEQ ID NO:1; nucleic acid sequence on bottom line is SEQ ID NO:2; P2-A cleavage site is SEQ ID NO:9; mIL4 insert is SEQ ID NO:10.

FIG. 2 shows the amino acid sequence of the PLS-CVB3 genome (SEQ ID NO:11) and the mIL-10-CVB3 (SEQ ID NO:12) at the site of the protease 2A cleavage. In this construct, the cloning procedure has been modified to include a polylinker site (PLS) to facilitate the use of the CVB3 as a generic cloning and expression vehicle. Further modifications include non-direct repeat genetic sequences to code for the protease P2-A cleavage site in the nascent polyprotein. The amino acids donated by the PLS are underlined, while the amino acids which form the 2A cleavage recognition signal are double underlined. The sequence of the mIL-10 insertion is shown in bold.

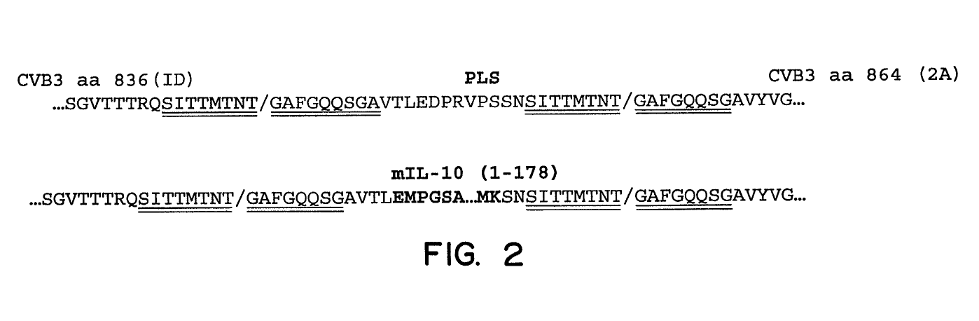

FIG. 3 shows the nucleotide and amino acid sequence of the PLS-CVB3 genome (SEQ ID NOS: 13 and 15) and mIL-10-CVB3 genome (SEQ ID NO:14, in which nucleotides 1-27 are SEQ ID NO:5 and nucleotides 28-55 are SEQ ID NO:6, and SEQ ID NO:16) at the beginning of the open reading frame. In this construct, the foreign or heterologous sequence is cloned in the open reading frame upstream of the first encoded viral protein. The translational initiation thus occurs at the beginning of the mIL10 sequence (or other sequence of interest). This construct employs either the viral protease 3C to cleave the foreign protein, here modeled as mIL10, from the first viral capsid protein P1-A. The nucleotide and amino acid sequence of the PLS are underlined and the protease 3C recognition site is double underlined. The sequence of the mIL-10 insertion is shown in bold.

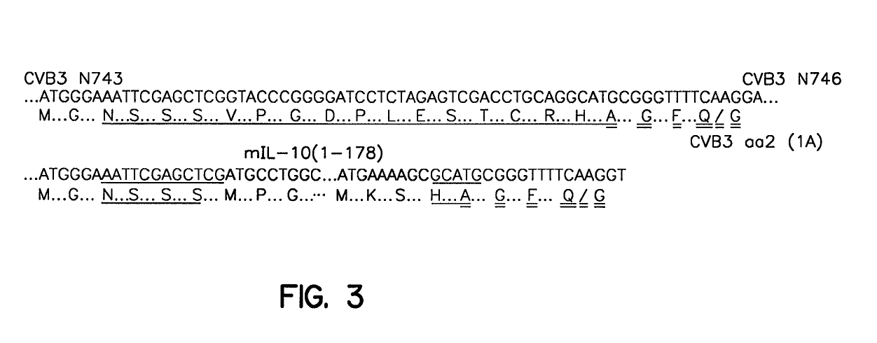

FIG. 4 shows the structure of the CPV/49-Polylinker genome. Nucleic acid sequence is SEQ ID NO:17 (nucleotides 1-60 and 62-75 comprise SEQ ID NO:7 and nucleotides 76-138 are SEQ ID NO:8; amino acid sequence is SEQ ID NO:18.

FIG. 5 shows the results of a slot blot of total RNA from HeLa cells inoculated with sequential passages of CVB3/0-IL4 and probed with (left) an mIL4-specific oligonucleotide or (right) a CVB3-specific oligonucleotide. After transfection of the pCVB3/0-IL4 cDNA into HeLa cells and obtaining progeny virus, a stock was made in HeLa cells (pass 1). The stock was used to inoculate a 100 mm dish of HeLa cells at an MOI of 20 (pass 2). After titering, pass 2 was used to inoculate new HeLa cells (pass 3), and so on. To obtain RNA for these experiments, passes 1-5 were used to inoculate a nearly confluent 100 mm dish of HeLa cells at an MOI of 20. Cells were washed after 1 hour, and harvested 5 hours post-infection. Total nucleic acids were digested with DNase. The equivalent of 2×10⁵ and 0.4×10⁵ cells were blotted for each passage. The same mass of oligonucleotide probe with equivalent specific radioactivities were used for each strip. Control blots using an alpha-tubulin probe demonstrated each RNA concentration used to be equivalent; RNase treatment of control blots demonstrated no RNA or DNA was detectable.

FIG. 6 shows histologic thin sections of murine pancreatic tissue following infection of mice either by CVB3/0 (left-hand panel) or CVB3/0-IL4 (right-hand panel). Mice were sacrificed on day 10 post-infection. CVB3/0 induces severe pancreatic acinar cell destruction; intact acinar cells can be seen in lower right hand corner of the panel. CVB3/0-IL4 does not induce any observable pathologic changes in the murine pancreas (right hand panel).

FIG. 7 shows construction of pCVB3-PL2-Ad2L1 as described in Example 5. FIG. 7a: the construction of the recombinant plasmid using a CVB3/0-derived subclone, pBSPL2, is shown. The CVB3 ORF is indicated by open bars, the Ad2 hexon L1 loop insert is indicated by a solid box, and the polylinker is indicated by a shaded box. Regions encoding 2Apro recognition sites are indicated by black arrows. NTR, nontranslated region. PolyA, poly(A) tract located at the 3′ end of the CVB3 genome. FIG. 7b: the nucleotide sequence of the polylinker with the flanking dissimilar duplicated region that encodes the modified 2Apro cleavage site (shaded bar) is given. Nucleotide numbering is based on the CVB3/0 genome found at GenBank accession no. M88483. The nucleotide sequence is SEQ ID NO:19; the amino acid sequence is SEQ ID NO:20.

FIG. 8 shows replication of CVB3-PL2-Ad2L1 in cell culture. FIG. 8a: single-step growth curves of CVB3-PL2-Ad2L1 and CVB3/0 in HeLa cells were obtained as described in the text. Cultures were harvested by freezing at the indicated times postinoculation. Virus titers were determined on HeLa cell monolayers and are expressed as the logarithm of TCID₅₀ per milliliter with each data point representing the mean titer and standard deviation of triplicate independent experiments. FIG. 8b: yields of infectious virus in MFHF, HCAEC, and COS-1 cultures 24 h after inoculation with CVB3-PL2-Ad2L1 or CVB3/0.

FIG. 9 shows western blot analysis of viral proteins in CVB3-PL2-Ad2L1-inoculated HeLa cells. Proteins were harvested 5 and 7 h postinoculation from HeLa cell monolayers inoculated with either CVB3-PL2-Ad2L1 or CVB3/0, separated by SDS-polyacrylamide gel electrophoresis, transferred to nitrocellulose, and probed with an anti-CVB3 antibody. Anti-CVB3 primary antibody was detected with a horseradish peroxidase-linked second antibody, and the signal was developed using an enhanced chemiluminescence system (ECL+; Amersham). Molecular masses at which the CVB3 capsid protein (CVB3-1D) and the chimeric protein (CVB3-1D/Ad2-L1) migrate are indicated.

FIG. 10 shows PCR and sequence analysis of CVB3-PL2-Ad2L1. pCVB3-PL2-Ad2L1 was transfected into HeLa cells, and the resultant progeny virus (CVB3-PL2-Ad2L1, pass 1) was subsequently serially passaged in HeLa cell cultures (passes 2 to 10). Viral RNA was isolated from virus stocks at each passage, and the presence of the inserted Ad2 sequence was analyzed by PCR using primers flanking the insertion site in the CVB3 genome. FIG. 10a: amplimers were separated by agarose gel electrophoresis. CVB3-PL2-Ad2L1, RT-PCR amplimer using chimeric viral RNA as the template; pCVB3-PL2-Ad2L₁, PCR amplimer using the chimeric plasmid DNA as the template; CVB3/0, RT-PCR amplimer using the parental CVB3/0 RNA as template; neg., RT-PCR using RNA as template from uninfected HeLa cells; Marker, 100-bp DNA ladder. FIGS. 10b and 10c: the sequence of the Ad2 insert-containing 446-bp amplimer (CVB3-PL2-Ad2L1) (FIG. 10b; nucleic acid sequence is SEQ ID NO:21; amino acid sequence is SEQ ID NO:22) and the sequence of the 225-bp Ad2 fragment-deleted amplimer (CVB3-PL2-Ad2L1del) (FIG. 10c; nucleotide sequence comprises bases 1-49 of SEQ ID NO:21; amino acid sequence comprises residues 1-16 of SEQ ID NO:22) were obtained after isolation of the DNA fragments from agarose gels. Sequence analysis was performed with the same primers as for the RT-PCR analysis. Numbering is based on the CVB3/0 genome (Genbank accession no. M88483).

FIG. 11 shows kinetics of CVB3-PL2-Ad2L1 replication in murine sera and pancreata. BALB/c mice were inoculated as described in the text with 5×10⁵ TCID₅₀ of CVB3-PL2-Ad2L1 per mouse and then sacrificed on days 1, 2, 4, 6, and 8 postinoculation. For each mouse, virus titers were measured in serum (TCID₅₀/milliliter), pancreas (TCID₅₀/gram), and heart (TCID₅₀/gram). The data are shown as mean and standard deviation for three animals per time point. No titer was measurable in any heart tissue.

FIG. 12 shows a diagram of the multivalent CVB vaccine construct. The capsid protien 1D BC loops from CVB2 (B2) and CVB4 (B4) were inserted into the CVB3/0-derived subclone, pBSPL2.

FIG. 13 is a graph showing that CVB3-expressed mIL-4 suppresses diabetes in NOD mice. X axis is age of mice in weeks, Y axis is percent of glycosuric mice in total population (glycosuria defined as>2000 mg glucose/dL blood).

FIG. 14 is a graph showing that inoculation of young NOD mice with CVB3 strains of varying virulence suppresses IDDM. X axis is age of mice in weeks, Y axis is percent of glycosuric mice in total population (glycosuria defined as>2000 mg glucose/dL blood).

### DETAILED DESCRIPTION OF THE INVENTION

The following definitions are used throughout the specification.

As used herein, the term “Coxsackie B3 virus;” or “CVB33” refers to a specific serotype of the human coxsackie B enterovirus of the family Picornaviridae, genus Eterovirus. The CVB3 genome is characterized by a single molecule of positive sense RNA which encodes a 2,185 amino acid polyprotein.

As used herein, the term “cardiotropic” refers to the targeting of heart tissue by a virus, in this case Coxsackievirus B3.

As used herein, the term “attenuated” refers to a virus, in this case Coxsackievirus B3, that is engineered to be less virulent (disease-causing) than wildtype Coxsackievirus B3.

As used herein, the term “one way viral vector” refers to viral delivery vehicles which are replication deficient for virus production but the RNA genomes of which can autonomously replicate in infected cells for variable periods of time. Such a vector permits replacement of essentially all of the capsid coding region with other sequences of interest, potentially delivering as many as seven cytokine-size coding sequences in the viral genomes. Such genomes made defective through deletion of a polymerase sequence and under a mammalian promoter may be used as a vector for a DNA vaccine or therapeutic, to be delivered by standard means, such as injection or oral administration.

As used herein, the term “basic CVB3/0 genome” shall mean the bioengineered Coxsackievirus B3 as reported by Chapman, N. M., et al, Arch. Virol. 122:399-409 (1994).

As used herein, the term “viral protease” or “viral encoded protease” refers to viral encoded enzymes that degrade proteins by hydrolyzing peptide bonds between amino residues. Some such proteases recognize and cleave at only specific sequences.

As used herein, the term “immunomodulatory gene” refers to a gene, the expression of which modulates the course of an immune reaction to a specific stimulus or a variety of stimuli. Examples include interleukin 4, interleukin 10, tumor necrosis factor a, etc.

As used herein, the term “cytokine” refers to a small protein produced by cells of the immune system that can affect and direct the course of an immune response to specific stimuli.

As used herein, the term “antigenic epitope” refers to a sequence of a protein that is recognized as antigenic by cells of the immune system and against which is then directed an immune response, such as an antibody response, for example.

As used herein, the term “viral vector” refers to a virus that is able to transmit foreign or heterologous genetic information to a host. This foreign genetic information may be translated into a protein product, but this is not a necessary requirement for the foreign information.

As used herein, the term “open reading frame” refers to a length of RNA sequence, between an AUG translation start signal and any one or more of the known termination codons, which can be translated potentially into a polypeptide sequence.

As used herein, the term “capsid coding region” refers to that region of a viral genome that contains the DNA or RNA code for protein subunits that are packaged into the protein coat of the virus particle.

In accordance with the present invention there may be employed conventional molecular biology, microbiology, and recombinant DNA techniques within the skill of the art. Such techniques are explained fully in the literature. See, e.g., Sambrook et al., “Molecular Cloning: A Laboratory Manual (1989); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984); or “Current Protocols in Molecular Biology”, eds. Frederick M. Ausubel et al., John Wiley & Sons, 1997.

Therefore, if appearing herein, the following terms shall have the definitions set out below.

A “vector” is a replicon, such as plasmid, phage, cosmid, or virus to which another DNA or RNA segment may be attached so as to bring about the replication of the attached segment. A vector is said to be “pharmacologically acceptable” if its administration can be tolerated by a recipient mammal. Such an agent is said to be administered in a “therapeutically effective amount” if the amount administered is physiologically significant. An agent is physiologically significant if its presence results in a change in the physiology of a recipient mammal. For example, in the treatment of retroviral infection, a compound which decreases the extent of infection or of physiologic damage due to infection, would be considered therapeutically effective.

An “origin of replication” refers to those DNA sequences that participate in the in the initiation of DNA synthesis.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease Si), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence.

As used herein, the terms “restriction endonucleases” and “restriction enzymes” refer to bacterial enzymes, each of which cut double-stranded DNA at or near a specific nucleotide sequence.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. In another example, coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

The present invention provides a viral vector for delivering a heterologous nucleic acid to a target cell, tissue or organ, which comprises a coxsackievirus genome modified to encode an attenuated coxsackievirus. The genome further comprises at least one cloning site for insertion of at least one expressible heterologous nucleic acid. Although in a preferred embodiment, the coxsackievirus genome is a coxsackievirus B genome, most preferably a coxsackievirus B3 genome, any coxsackievirus genome is believed to be suitable for use in the present invention. This is due to the high level of organizational similarity among the coxsackieviruses, and indeed among enteroviruses in general (see, e.g., Romero et al., Current Topics in Microbiology and Immunology 223: 97-152, 1997, reviewing the genetic relationship among the Group B coxsackieviruses; Chapman et al., Current Topics in Microbiology and Immunology 223: 227-258, 1997, reviewing the genetics of coxsackievirus virulence; and Tracy et al., Trends in Microbiology 4: 175-179, 1996, reviewing the genetics of coxsackievirus B cardiovirulence and inflammatory heart muscle disease). Thus, in the present invention it has been demonstrated that CVB3 can be attenuated by manipulation of the genome in a variety of ways, most of them relating to altering a transcription regulatory region of the genome, such as the 5′ untranslated region of the genome. These alterations are described in greater detail below, and in the examples. Similar manipulations likewise can be made to attenuate any of the other five coxsackievirus B serotypes, as well as coxsackievirus A or other enteroviruses.

It has also been demonstrated in accordance with the present invention that a heterologous DNA segment can be inserted in the CVB3 genome in one of several locations, e.g., between a coding sequence for a capsid protein and a coding sequence for viral protease, or at the start of the genome's open reading frame, in such a manner that the heterologous DNA comprises a translation start codon and a 3′ sequence recognized by a viral protease. These insertions are described in greater detail below, and in the Examples. Similar insertions likewise can be made in the genomes of the other coxsackieviruses or other enteroviruses, and successful expression of these heterologous nucleic acids also is expected. Moreover, one skilled in the art also will appreciate that other useful insertion sites exist and can be exploited in the coxsackievirus genome.

Concerning the size of the heterologous nucleic acid that can be inserted into a coxsackievirus vector of the invention, it has been discovered that the genome can incorporate an insert encoding up to 200-400 amino acids. The size of the insert may be increased (e.g., to inserts encoding 800-1,000 amino acids), if certain portions of the genome (e.g., capsid protein coding sequences) are deleted. In this embodiment, it would be necessary to supply a helper virus to provide the missing capsid proteins in trans, for packaging the virus. Such manipulations of viral vectors are well known to persons skilled in the art.

The heterologous nucleic acid sequence carried by the coxsackievirus vector of the invention can encode any gene product, including RNA of any kind, peptides and proteins. In one embodiment, the expressible heterologous DNA carried by the coxsackievirus vector of the invention encodes an antigenic product. In another embodiment, it encodes a biologically active product, such as a biologically active protein. Preferably, the protein is a cytokine, such as IL-4 or IL-10, as described in greater detail below and in the examples.

Particularly preferred aspects of the present invention are directed to a bioengineered virus for the therapeutic delivery of at least one heterologous gene to a target organ or organ system in an individual, comprising a Coxsackievirus B3, wherein said Coxsackievirus B3 is cardiotropic and attenuated, and wherein the genome of the CVB3 codes for the at least one heterologous gene.

It is contemplated additionally that the present invention provides (1) a method for vaccinating an individual, comprising administering a coxsackievirus containing a gene for an antigenic epitope to an individual, and (2) a method for suppressing an immune response in an individual, comprising administering the coxsackievirus vector containing an immunomodulatory gene to an individual.

With respect to using the coxsackievirus vectors of the invention to vaccinate an individual, the inventors have demonstrated that a vector of the invention stably expresses an antigenic polypeptide of Adenovirus 2 (Ad2) from within the CVB open reading frame that results in the induction of protective immune responses against both CVB3 and Ad2. As described in detail in Example 5, the inventors cloned the sequence encoding the Ad2 hexon L1 loop, flanked by dissimilar sequences encoding the protease 2A (2Apro) recognition sites, into the genome of an attenuated strain of CVB type 3 (CVB3/0) at the junction of 2Apro and the capsid protein 1D. Progeny virus (CVB3-PL2-Ad2L1) was obtained following transfection of the construct into HeLa cells. The Ad2 hexon L1 loop and flanking amino acids were expressed from within the ORF of CvB3/0. The inserted Ad2 coding sequence affected the yield of CVB3-PL2-Ad2L1 relative to the parental virus, but it was maintained stably in the vector RNA through at least 10 generations in HeLa cell cultures. The chimeric virus replicated in mice and presented the Ad2 polypeptide to the immune system as demonstrated by the induction of both anti-Ad2 neutralizing and binding antibodies. The chimeric CVB3-based virus induced anti-Ad2 immunity in mice with preexisting anti-CVB3 immunity.

In similar experiments, it was further shown that a multi-insert CVB3 vector of the invention comprising CVB2 and CVB4 antigen encoding inserts was able to induce neutralizing antibodies against CVB2, CVB3 and CVB4. Thus, the vectors of the invention can be used to produce multivalent vaccines against viruses or other infectious agents.

This utility of the present vectors for vaccines has a wide range of applications, inasmuch as it permits not only multiple vaccine targets with a single vector, but it also permits repeated vaccinations for the treatment of disease. For instance, in AIDS, decreasing the virus load is part of the treatment for the disease. Designer vaccines can be tailored for individuals and their own virus populations to vaccinate against newly-arisen populations in individual patients.

For gene delivery applications, a person having ordinary skill in the art of molecular biology, gene therapy and pharmacology would be able to determine, without undue experimentation, the appropriate dosages and routes of administration of the novel coxsackievirus gene delivery vector of the present invention.

One specific object of the present invention is to use artificially attenuated cardiotropic virus vectors as efficient gene transfer vectors to deliver immunomodulatory proteins and/or antigenic epitopes in transient infections to aid in preventing, ameliorating, and/or ablating infectious viral heart disease. The invention encompasses reducing, or ablating entirely, heart transplant rejection through therapeutic use of immunosuppressive cytokines delivered by attenuated cardiotropic virus vectors. The invention is equally applicable to other inflammatory diseases or conditions of a variety of organs. In this aspect, the invention thus requires three elements: First, an attenuated CVB3 viral vector must be provided. Second, the CVB3 viral vector must be able to express an immunomodulatory protein, such as a cytokine. Third, the vector must be able to deliver the immunomodulatory protein to the target tissue and observably reduce disease symptoms. These three elements are provided in the present invention. Cardiovirulence of CVB3 has been reduced to complete attenuation for heart disease by the substitution of the entire 5′ NTR with that of a non-coxsackie enterovirus. The murine cytokine IL-4 (mIL-4) has been expressed within the open reading frame of an attenuated CVB3 strain and has been demonstrated to be biologically active. Inoculation of the CVB3 chimera expressing mIL-4 into mice 1 or 3 days post-inoculation with a pancreovirulent CVB4 strain significantly ablates CVB4-induced pancreatic disease. These data exemplify the unique therapeutic approach to inflammatory diseases of the present invention.

Another aspect of the present invention relates to vectors and vaccines for the prevention and/or treatment of insulin-dependent (type 1) diabetes mellitus (IDDM). IDDM is a chronic disease characterized by an autoimmune, predominantly Th1, response against pancreatic beta cells. It has been shown experimentally that the onset of IDDM may be delayed or reduced by repeated administration of certain cytokines, such as IL-4. Presumably, the mechanism by which this occurs is related to induction by the cytokine of a Th1 Th2 isotype shift. As described in Example 7, the present inventors have now demonstrated that inoculation with a coxsackievirus of the invention encoding IL-4 induces the same effect in a non-obese diabetic (NOD) mouse model, thereby protecting the animals from the onset of IDDM. It was also shown that inoculation of young animals with various strains of CVB3 alone (not encoding a heterologous polypeptide) resulted in suppression of the onset of IDDM in the NOD mouse model (Example 8). Thus, the attenuated coxsackievirus vectors of the present invention are useful for the treatment or prevention of IDDM.

The following examples are set forth to illustrate various embodiments of the invention and are not meant to limit it in any fashion.

### EXAMPLE 1

### Artificial Attenuation of CVB3 for Cardiac Disease in Mice

It has been demonstrated that 5′ NTRs of related enteroviruses could be exchanged and viable progeny virus produced when a poliovirus type 1 5′ NTR was replaced with some or all of a CVB3 5′ NTR (Johnson V. H., and B. L. Semler, Virology 162(1):47-57 (1988); and Semler B. L., et al., Proc-Natl Acad Sci USA 83(6): 1777-81 (1986)). For the present invention, a variety of CVB3 strains with genomes chimeric in the 5′ and/or 3′ non-translated regions (NTR) sequences has been constructed from poliovirus type 1. The construct that consists of the 5′ NTR from PV1/Mahoney and the remainder of the genome from CVB3/20 has been used most extensively in the investigation of the current invention.

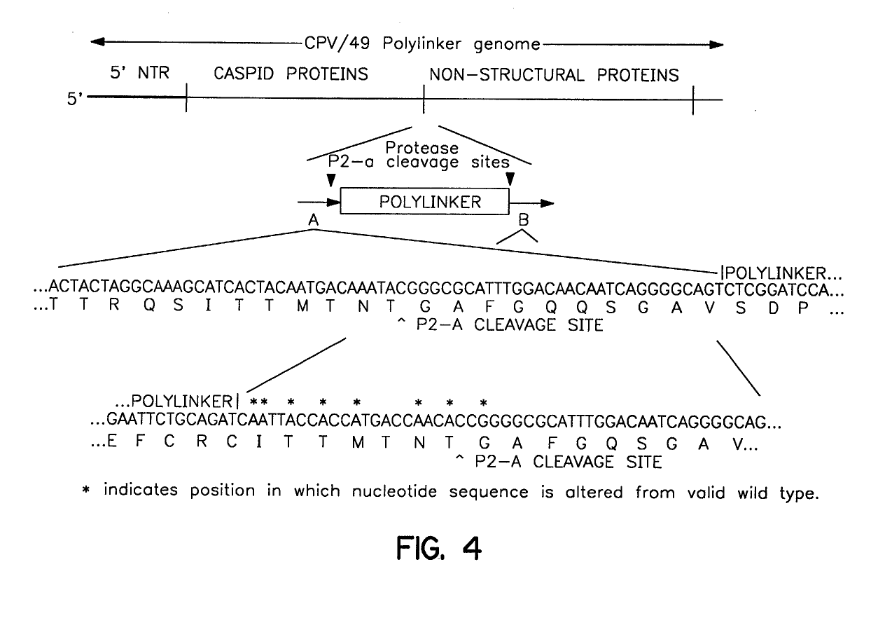

Five passages of this chimeric virus, CPV/49 (FIG. 4), did not result in genetic alteration in the donated poliovirus 5′ NTR on the basis of sequence analysis. Replacement of a cardiovirulent CVB3 5′ NTR with the homolog from the neurovirulent PV1 Mahoney strain results in a progeny virus that is (a) genetically stable in cell culture in terms of maintaining the PV sequence of the 5′ NTR; and (b) highly attenuated for its ability to induce myocarditis in mice, and replicates to 3-4 logs lower titer in the murine heart relative to the parental cardiovirulent CVB3/20 strain. Notwithstanding this attenuation, antibody titers are induced against CVD3 in the inoculated mice that prevent cardiac disease when the mice are challenged with inoculation by a cardiovirulent CVB3 strain.

These data demonstrate that a CVB3 virus strain made chimeric with the replacement of the 5′ NTR from PV1 results in a CVB3 strain that is stably attenuated for heart disease when measured in mice and animals, and, furthermore, acts as a vaccine strain by preventing heart disease due to challenge by cardiovirulent CVB3 infection. Thus, such a virus strain acts as a delivery system as envisioned in the present invention.

In addition, the mechanism by which a non-cardiovirulent CVB3 strain (CVB3/0) is attenuated for cardiovirulence has been mapped and identified. By comparison of the complete nucleotide sequences of the avirulent and cardiovirulent CVB3 strains and analyzing a series of intratypic chimeric viruses designed to test the potential genetic sites, a single site nt234 was demonstrated to be the sole site that affected cardiovirulence in these virus strains (Tu Z., et al., J Virol 69:4607-18(1995)). The nt234 is U in the cardiovirulent strain, and C in the avirulent strain. Assay in murine heart cells demonstrated little or no detectable differences in Western blotted viral proteins between the two strains, but at least a ten-fold disparity in viral RNA transcription rate was identified. Further work has shown that the normally high positive to negative viral RNA strand ratio in infected cells is significantly altered to near unity when nt234 is C rather than U.

Two further observations make it clear that alteration of certain 5′ NTR sequences results in attenuation. One is that mutation of nt234U to G also results in attenuation by what appears to be a similar mechanism to that observed for nt234 C. Second, mutation of this same nucleotide to G in PV1/Mahoney also results in a strain of virus that grows less robustly in HeLa cells than the parental virus. Because nt234 is conserved as U in all enteroviral RNAs examined so far (Chapman N. M., et al., J. Med. Virol. 52: 258-261, 1997), as are the surrounding 5 nucleotides 5′-CGUUA (nt234 is underlined), mutation at this site appears to be generally deleterious for enterovirus health. A CVB3 strain, chimeric in the 5′ NTR using the PV1 sequence with the added mutation of G instead of U at the PV equivalent of nt234 provides a stably attenuated (but possibly quite weak) CVB3 strain, even less prone to reversion to cardiovirulence than the stably attenuated CVB3/PV1 chimeric described above. Either of these chimeric CVB3 strains is suitable for the viral delivery vector of the present invention in which murine interleukins are expressed within the open reading frame of an artificially attenuated CVB3 strain.

### EXAMPLE 2

### Successful Expression of Biologically Active Murine IL-4 from Within the CVB3 Open Reading Frame

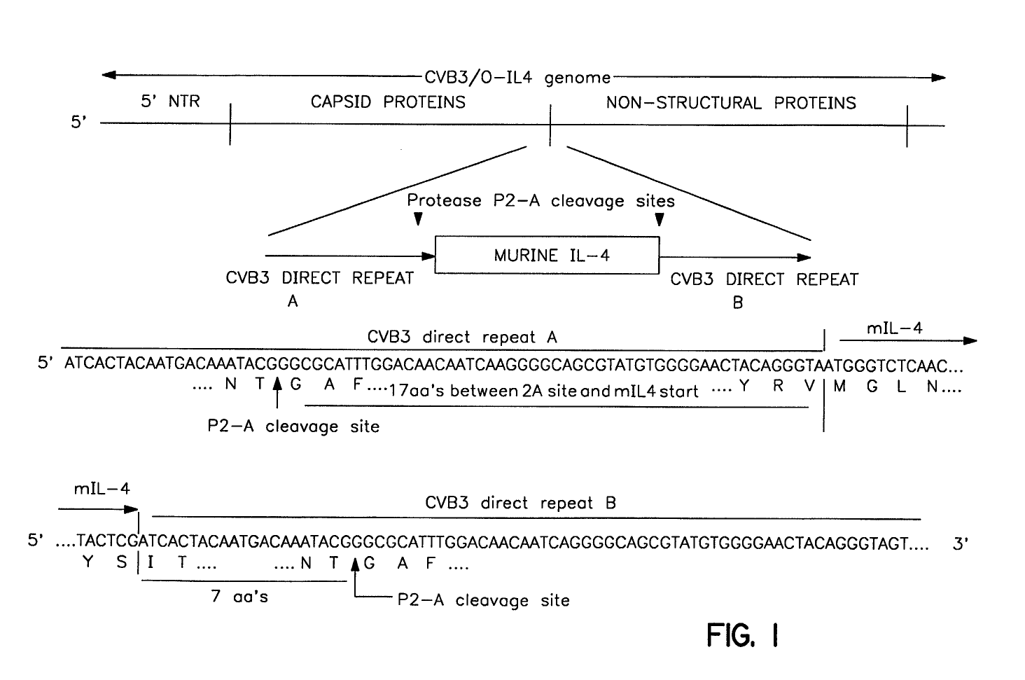

One viral vector construct envisioned by the present invention is depicted in FIG. 1. Acute rejection of a transplanted heart involves primarily a Th1 type T cell response, the same type of T cell response that is observed in CVB3 induction of acute myocarditis in well-studied murine models of CVB 3-induced inflammatory heart disease. Switching of the response to the Th2 type response causes a concomitant ablation of disease. Due to the interest in increasing Th2 type responses, expression of the murine IL-4 gene (mIL-4) was chosen. The virus vector used was the infectious cDNA clone of CVB3/0, a CVB3 strain effectively attenuated for murine heart disease through the mutation at nt234 (from U to C). The mIL-4 sequence contained the signal sequence to facilitate extracellular transport of the expressed interleukin protein (see Sideras P., et al., Adv Exp Med Biol 213:227-23.6 (1987)). Flanking the mIL-4 insert were cloned identical sequences that are recognized by the CVB3 protease 2A. The mIL-4 insert plus the flanking sequences encoding the protease 2A recognition cleavage sites were cloned in-frame at the junction of the capsid protein 1D and protease 2A.

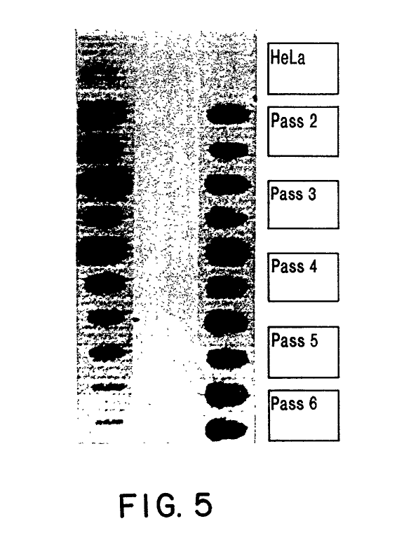

The construct gave rise to progeny virus (termed CVB3/0-IL4) when electroporated into HeLa cells. Sequence analysis by reverse-transcriptase mediated PCR followed by sequence analysis of the amplimer confirmed that the progeny virus contained the insert and that the viral open reading frame was maintained. The mIL-4 coding sequence in the viral RNA was detected readily by slot blot analysis through 5 passages in HeLa cells, after which deletion occurs rapidly (FIG. 5). This is most likely due to recombination in the 72 nucleotide direct repeat that was engineered to duplicate the protease 2A cleavage sites (see FIG. 1). This is not unexpected: once a CVB3/0 genome deletes the mIl-4 coding sequence, it would be expected to replicate more rapidly, and would rapidly become the dominant quasi species. This may be reflected in the blot following the CVB3 RNA as well: later passages suggest slightly more viral RNA present in the samples.

That the strain CVB3/0-IL4 expressed murine IL-4 in HeLa cells was confirmed by ELISA. Virus was inoculated onto HeLa cells, excess virus was removed by washing at one hour post infection, and the cells were re-fed. At times post-inoculation, the supernatant was removed and then the cells were frozen in a similar volume of fresh medium. Following freezing and thawing and removal of cell debris by centrifugation, the cell medium samples, and the cell fractions were assayed using a commercially available ELISA test for murine IL-4 (BioSource International, Inc.). CVB3/0-IL4 produced mIL-4 intracellularly well above the uninfected control background, reaching 300 pg/ml by 6 hours in cultures producing 106 TCID₅₀ units of virus/ml.

Biological activity of the CVB3/0-IL4 expressed murine IL-4 was assessed using supernatants from HeLa cells infected with the virus, washed with media, incubated for 6-8 hours, then frozen and thawed. Supernatants cleared of cellular debris were assayed for ability to induce MC/9 mouse mast cells to proliferate using an MTT assay (Mosmann T., J Immunol Methods 65(1-2):55-63 (1983); and Gieni S, et al., J Immunol Methods 187(1):85-93 (1995)) with recombinant mIL-4 as standard. CVB3/0-IL4 HeLa cultures produced 3 units/ml (equivalent to 250 pg/ml of recombinant mIL-4). This compares favorably with reported IL-4 levels in coronary sinus blood concentrations in cardiac transplant patient (229 pg/ml; Fyfe A, et al., J Am Coll Cardiol 21(1):171-6 (1993)).

### EXAMPLE 3

### Diminution of CVB4-Induced Pancreatic Disease in Mice by Treatment with mIL-4 Expressing CVB3

In an initial test of the ability of the CVB3-1L4 strain to decrease inflammatory disease induced by enteroviruses, a virulent CVB4 strain was used as the inflammatory disease inducer. A different CVB serotype was chosen to minimize the possibility that neutralizing antibodies might reduce the replication of CVB3-1L4 in the doubly-infected mouse (Beck M., et al., Am. J. Pathol. 136:669-681 (1990)). The strain of CVB4, termed CVB4-V, was derived by repeated passaging in mice of the avirulent strain, CVB4/P until the virus was repeatedly able to induce severe destruction of the murine pancreatic acinar cells (Ramsingh A., et al, Virus Res 23(3):281-92 (1992)). The pancreatic disease induced by this virus is likely to have an immune component based on the lack of correlation between virulence and the extent of virus replication in the pancreas and the dependence upon host genetic background. Further, it has been demonstrated that CVB4/v is also pancreovirulent in C3H/HeJ male mice, the mice routinely employed to study CVB3 inflammatory heart disease (see Kiel R. J., et al., European Journal of Epidemiology 5:348-350 (1989)). In order to determine whether CVB3/0-IL4 would have an effect upon pancreatic disease induced by this strain of CVB4, the experiment outlined in Table 1 was performed.

TABLE 1
OUTLINE OF CVB4/CVB3 EXPERIMENT AND RESULTS IN
DISEASE/TOTAL PANCREASES OBSERVED AT DAY 10 PI

| | | | NUMBER | DAY 10 |
|---|---|---|---|---|
| DAY 0 | DAY 1 | DAY 3 | OF | PANCREATIC |
| INOC. | INOC. | INOC. | MICE | DISEASE |
| MEDIUM | NONE | NONE | 3 | NONE (3) |
| CVB3/0 | NONE | NONE | 4 | SLIGHT (1) |
| | | | | SEVERE (3) |
| CVB3/0-IL4 | NONE | NONE | 8 | NONE (7) |
| | | | | SLIGHT (1) |
| CVB4/V | NONE | NONE | 5 | SEVERE (5) |
| CVB4/V | CVB 3/0 | NONE | 5 | MODERATE (1) |
| | | | | SEVERE (4) |
| CVB4/V | NONE | CVB3/0 | 4 | SEVERE (4) |
| CVB4/V | CVB3/0-IL4 | NONE | 9 | SLIGHT (2) |
| | | | | MODERATE 5 |
| | | | | SEVERE (2) |
| CVB4/V | NONE | CVB3/0-IL4 | 10 | SLIGHT (2) |
| | | | | MODERATE (4) |
| | | | | SEVERE (4) |

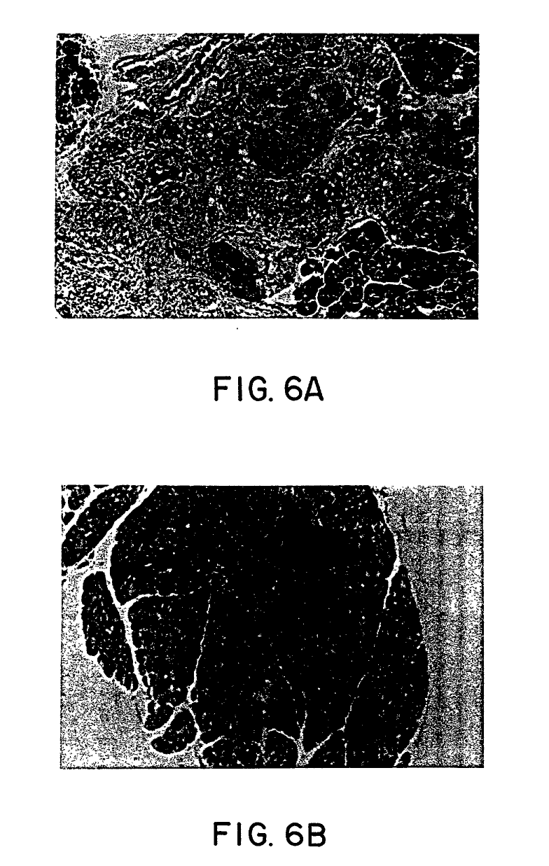

Briefly, mice were inoculated with 5×10⁵ TCID50 units of CVB4/V in 0.1 ml unsupplemented medium. One or three days later, mice were also inoculated with an equivalent dose of CVB3/IL4 (second passage virus stock after transfection). Control mice were inoculated with the parental (without IL-4 insert and 2A-cleavage site insert) CVB3/0 at the same times. In addition, mice were inoculated with unsupplemented medium without virus or with a single virus: CVB3/IL4, CVB4/V, or CVB3/0. On day 10 post-infection, pancreata were fixed in formalin, sectioned, stained with hematoxylin and eosin, and examined microscopically. Examples of the type of pathologies observed are shown in FIG. 6.

All the mice inoculated only with CVB4/V incurred massive pancreatic damage (Table 1). Mice inoculated with CVB4/V, and that subsequently received CVB3/0-IL4 either on day 1 or day 3 post-infection, demonstrated a significant ablation in the extent of disease. No significant difference was observed between pancreas tissue from mice with day 1 or day 3 post-infection (post CVB4/V) inoculation with CVB3/0-IL4. Mice that were inoculated with CVB4/V and subsequently inoculated with the attenuated parental CVB3/0 strain at either day 1 or 3, demonstrated pancreata that were indistinguishable from the CVB4/V only mice. Thus, the diminution of pancreatic damage observed in mice that received first pancreovirulent CVB4/V, then CVB3/0-IL4 on day 1 or 3 post infection, is due to the expression of the mIL-4 in the chimeric CVB3 strain.

In addition, the CVB3/0-IL4 construct was not virulent for the pancreas. Even though CVB3/0 is completely attenuated for heart disease, it causes significant and widespread destruction of the murine acinar cells. While mice that received only CVB3/0 demonstrated significant pancreatic damage, it is worth noting that the presence of the mIL-4 coding sequence in the CVB3/0 genome resulted in a virus which did not induce pancreatic disease in mice. These data, combined with the data above that showed a diminution of CVB4-caused pancreatic disease by administration of the CVB3/0-IL4 chimera, are consistent with a beneficial role upon pancreatic disease diminution caused by an enterovirus.

### EXAMPLE 4

### Attenuation of Coxsackievirus B3 Replication by Two Point Mutations in the 5′ Non-translated Region

In Example 1 we described a conserved 5-nucleotide region, surrounding nt234 of the CVB3 genome, that appears important for replication of the enterovirus genome. In this Example, the molecular grounds for the complete conservation of that 5′-CGUUA (nt 232-236) in the enteroviral 5′ non-translated region are examined. Using the well-characterized enterovirus model system, CVB3, point mutations were created at nt233 (GSC) and nt236 (A-U) in the CVB3 5′ non-translated region using site specific mutagenesis, according to standard methodology. This double mutant (pCVB3-88) was electroporated into HeLa cells and the progeny virus (CVB3/88) was passaged six consecutive times in HeLa cells. Virus from each passage was assayed in single-step growth curves and by nucleotide sequence analysis.

Prior to passage 3, CVB3/88 was highly attenuated, generating barely detectable titers. Passage 3 CVB3/88 entered log phase replication 3 hours later and achieved final titer 100 fold lower than the parental (control) CVB3 strain. Passage 4 showed an improved rate of replication and final titer 10 fold lower than the parental virus. CVB3/88 passage 5 replication was essentially indistinguishable from the parental strain.

Direct sequence analysis of CVB3/88 RNA using RT-PCR demonstrated that complete reversion had occurred by passage 5, whereas passage 4 virus indicated a partial reversion at nt233(G/C) and complete reversion at nt236 (U-A). Passage 3 showed partial reversion at both sites.

Reacquisition of wild-type replication rate and efficiency is directly correlated with reversion of the mutations to wild-type sequence. The degree of initial attenuation, and concomitant rapidity of reversion argues against robust compensatory mutations arising elsewhere in the viral genome, and is consistent with the previous evidence that this 5 nucleotide tract is absolutely conserved for efficient enteroviral replication.

It should be noted that live, attenuated viruses are useful as vaccines or gene delivery vehicles even if they revert to wild-type through several passages in cultured cells. In fact, live attenuated polioviruses exhibit reversion to wild-type, and these have been used as highly successful oral vaccines for many years. The risk of reversion after a single administration to a living individual (as opposed to several passages in cultured cells) is low, due to the fact that a normal individual will mount an immune response to the virus and clear it from the system before it has the opportunity to replicate to pathogenic levels in a critical target tissue (e.g., neurons). As a result, live, attenuated poliovirus is an effective vaccine even though it reverts to wild-type after passaging through culture cells. Likewise, forms of live, attenuated coxsackievirus and other enteroviruses that may revert to wild-type in culture still will be effective and useful for a variety of purposes. Less reversion-prone viruses, such as the CPV/49 described in Example 1, could be used for purposes where a reversion-prone attenuated virus is inappropriate.

### EXAMPLE 5

### Expression of an Antigenic Adenovirus Epitope in a Group B Coxsackievirus

In this example we describe an attenuated chimeric CVB3 strain that stably expresses the antigenic Li loop of the Ad2 hexon protein (Toogood, C. et al., J. Gen. Virol. 73: 1429-1435) at the junction of the CVB3 capsid protein 1D and the 2Apro. The progeny chimeric virus overexpresses viral protein, replicates, and induces neutralizing and binding anti-Ad2 antibodies, as well as anti-CVB3 neutralizing antibodies, in mice. Anti-Ad2 immunity can be induced in the presence of preexisting murine anti-CVB3 immunity.

Materials and Methods

Cells and viruses. Monolayer cultures of HeLa cells as well as cultures of murine fetal heart fibroblasts (MFHF) and COS-1 cells were propagated in minimal essential medium containing 10% fetal bovine serum and 50 ug of gentamicin per ml. Human cardiac artery endothelial cell (HCAEC) cultures were obtained from Clonetics (Walkersville, Md.) and were propagated as monolayers, as suggested by the supplier, in proprietary medium purchased from Clonetics. The cells were grown at 37° C. in a humidified 5% CO₂-air mixture. The infectious cDNA copy of the CVB3/0 genome (described herein and found at GenBank Accession No. M88483) was used as the background for the construction of the Ad2 hexon L1 loop polypeptide-expressing chimeric strain, CVB3-PL2-Ad2L1. Human Ad2 (American Type Culture Collection [ATCC], Manassas, Va.) and CVB3 strains were passaged in HeLa cell monolayers to produce virus stocks.

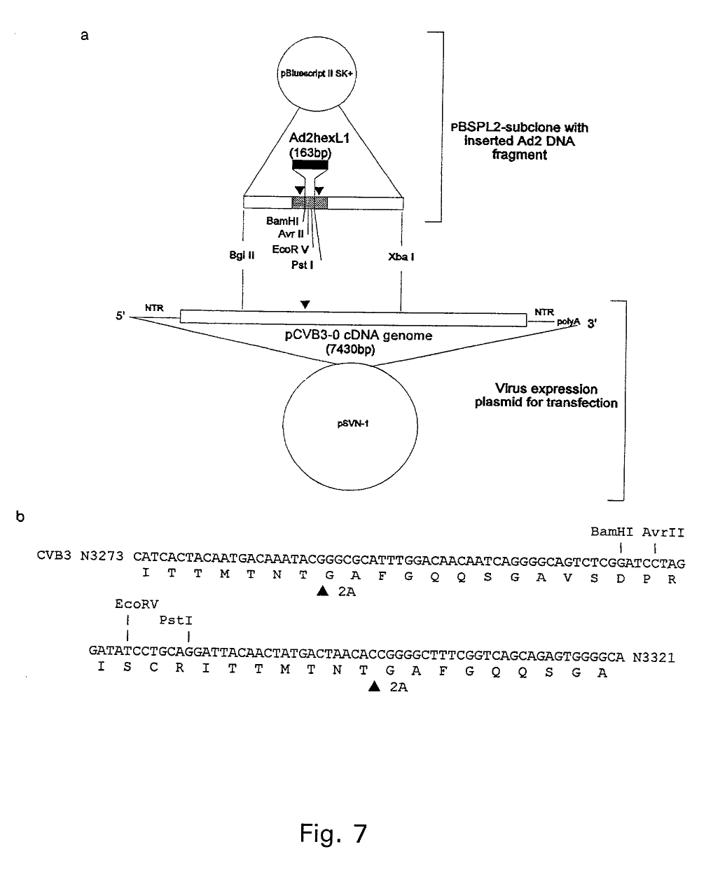

Construction and transfection of CVB3-PL2-Ad2L1. The construction of the infectious CVB3-PL2-Ad2L1 cDNA is outlined in FIG. 7a. For insertion of the Ad2 polypeptide encoded sequence, a CVB3/0-based subclone, pBSPL2, with a polylinker containing BamHI, AvrII, EcoRV, andPstI sites and flanked by a short sequence encoding the cleavage site of 2Apro was generated. Rather than flank an inserted sequence with perfect nucleotide repeats encoding the 2Apro recognition sites, changes were incorporated in the nucleotide sequence encoding the 2Apro cleavage site downstream of the insert that resulted in a sequence 49 nucleotides (nt) long with 69% similarity to the upstream wild-type site (FIG. 7b). This modified cDNA was first generated by PCR amplification using the pCVB3-0 cDNA as template with primers containing the altered nucleotide sequence with restriction sites and then inserted in a subcloned fragment of the pCVB3-0 cDNA (defined by the AvrII-ScaI fragment between nt 2034 and 5137 [numbering from GenBank accession no. M33854] cloned in the XbaI and EcoRV sites of pBluescript II SK+ (Stratagene, La Jolla, Calif.).

Two primers, HexA and HexD, (5′-TCCGGATGAAAAA GGGGTGCCTCTTCCAAAG, SEQ ID NO:23 and 5′-GCCTCT GCAGTCAGACAGATGTGTGTCTGG, SEQ ID NO:24, respectively), were used to amplify the L1 loop region from Ad2 DNA (Genbank locus ADRCG, nt 19624 to 19776); this fragment added a BamHI restriction site upstream and a PstI site downstream in frame with the CVB3 ORF. Cleavage at these two sites generated a fragment that was subsequently ligated into sublone pBSPL2 using the polylinker sites. The Ad2 insert-containing subclone was ligated into the pCVB3/0 cDNA genome using the uniqueBglII (nt 2042) and XbaI (nt 4947) sites. Sequence analysis of the resulting chimeric cDNA, pCVB3-PL2-Ad2L1, verified the existence of the Ad2-L1 loop coding sequence in frame with the CVB3 ORF.

To generate progeny virus, 3.5 μg of pCVB3-PL2-Ad2L1 were transfected into 3×10⁵ HeLa cells in a six-well plate using an Effectene transfection reagent kit (Qiagen, Valencia, Calif.) as suggested by the supplier. Three days posttransfection, cultures were frozen and thawed three times and centrifugally cleared of cell debris. One-third of the cleared supernatant was used to inoculate HeLa cells to obtain a CVB3-PL2-Ad2L1 virus stock (passage 2). Progeny virus was subjected to titer determination on HeLa cell monolayers and stored aliquoted at 74° C.

RT-PCR and sequence analysis. Total RNA was extracted from virus-infected cells (RNAzol; Life Technologies, Gaithersburg, Md.) and cDNA was synthesized using a one-step RT-PCR system as directed by the supplier (Superscript One-Step RT-PCR system; Life Technologies). The RNA sequence of pCVB3-PL2-Ad2L1 RNA across the cloning site was deduced by cycle sequencing of the resulting amplimers (ThermoSequenase; Amersham Life Science, Cleveland, Ohio). Enzymatic amplifications were performed for 40 cycles at an annealing temperature of 57° C. using primers ID9 and DI4 (5′-CTAGACTCTGCCAATACGAG [nt 3201 to 3220;SEQ ID NO:25] and 5′-GTGCTCACTAAGAGGTCTCTG [nt 3406 to 3426; SEQ ID NO:26], respectively). Nucleotide numbering is based upon the CVB3 sequence (accession no. M88483).

Single-step growth curves. Replication of the chimeric strain was compared to that of the parental strain using single-step growth curves as described by Tu et al. (1995, supra). Briefly, HeLa cells were inoculated at a multiplicity of infection of 20. After washing and refeeding of the cell monolayers, cultures were frozen at various times, thawed, and subjected to titer determination on HeLa cell monolayers for infectious virus.

Western blot analysis of viral proteins in infected cells. Translation of CVB3 proteins was studied by Western blot analysis of whole-cell virus-inoculated lysates basically as described by Chapman et al. (J. Virol. 74: 4047-4056). HeLa or MFHF cultures were inoculated with virus at a multiplicity of infection of 20 or s50, respectively, and the monolayer cultures were lysed at various times postinoculation with Laemmli buffer containing 2-mercaptoethanol. Proteins were electrophoresed in 14% acrylamide gels containing sodium dodecyl sulfate (SDS) (Novex, San Diego, Calif.) and electroblotted to Immobilon-P nylon membranes (Millipore, Bedford, Mass.). The blots were probed with a 1/1,000 dilution of the primary antibody, a polyclonal horse anti-CVB3 neutralizing antibody (ATCC) that detects CVB3 capsid protein 1D. The primary antibody was detected using peroxidase-conjugated rabbit anti-horse immunoglobulin G (IgG) (Jackson ImmunoResearch Laboratories, West Grove, Pa.) at a dilution of 1/125,000. The second antibody was detected using an ECL⁺kit and Hyperfilm (Amersham, Arlington Heights, Ill.) as directed by the supplier. A NucleoVision gel documentation system and software (Nucleo Tech, San Mateo, Calif.) were used to capture and analyze the X-ray film images.

Inoculation of mice with virus. Male BALB/c mice (Jackson Laboratory, Bar Harbor, Me.) were obtained at 3 to 4 weeks of age. The mice were inoculated intraperitoneally with 5×10⁵ 50% tissue culture infective doses TCID₅₀of either CVB3-PL2-Ad2L1 or the control CVB3/0 strain in 0.1 ml of unsupplemented medium. To determine whether the chimeric virus replicated in mice, mice were sacrificed 1, 2, 4, 6, or 8 days postinoculation and murine sera, pancreata, and hearts were obtained to measure virus titers in tissue as described elsewhere (Tu et al., 1995, supra). To determine whether the chimeric virus induced antiviral antibodies, mice were inoculated once, twice (14 days apart), or three times (each 14 days apart). Mice in each series were sacrificed 14 days following the final inoculation, and blood, hearts, and pancreata were removed for analysis. Longer times postinoculation were not investigated. Sera were isolated from clotted blood samples and stored frozen until use. For histopathologic analysis, hearts and pancreata were fixed in 10% buffered formalin, embedded, sectioned, and stained with eosin and hemotoxylin (Tu et al., 1995, supra). For studies of immunity in preimmune mice, cvB3/0 was inoculated using 5×10⁵TCID₅₀/0.1 ml intraperitoneally once or twice (14 days apart). Fourteen days later, one group of five randomly chosen mice were sacrificed and the pooled serum was tested for the presence of anti-CVB3 neutralizing antibodies. Two other groups of mice were challenged twice (14 days apart) with 5×10⁵TCID₅₀ of CVB3-PL2-Ad2L1 (CVB3/0-1x, Ad2-CVB3/0-2x or (CVB3/0-2x, Ad2-CVB3/0-2x) (Table 2). The mice were sacrificed 14 days after the final inoculation to obtain murine serum.

TABLE 2
Antibody response to CVB3-PL2-Ad2L2 infection
in mice^{a}

| | Virus-neutralizing titer^{c} | | Virus-binding |
|---|---|---|---|
| Antiserum^{b} | CVB3/0 | Ad2 | titer^{d} (Ad2) |
| CVB3-PL2-Ad2L1-1x | 1/16 | <1/2 | 1/20 |
| CVB3-PL2-Ad2L1-2x | 1/32 | 1/4 | 1/100 |
| CVB3-PL2-Ad2L1-3x | 1/64 | 1/8 - 1/16 | 1/1,000 |
| CVB3/0-1x and CVB3- | 1/128 | 1/16 - 1/32 | 1/5,000-1/10,000 |
| PL2-AD2L1-2x | | | |
| CVB3/0-2x and CVB3- | 1/128 | 1/32 | 1/10,000 |
| PL2-AD2L1-2x | | | |
| Hyperimmune CVB3 | >1/1,000 | | |
| Hyperimmune Ad2 | | >1/1,000 | >1/1,000 |

^{a}Mice were inoculated once (CVB3-PL2-AD2L-1x), twice (CVB3-PL2-Ad2L1-2x; 14 days apart), or three times (CVB3-PL2-Ad2L1-3x; each 14 days apart) with 5 × TCID₅₀ of virus. For studies of anti-Ad2 responses in CVB3/0-immunized mice (inoculated once or twice, 14 days apart) , mice were challenged twice with CVB3-PL2-A2dL1.
^{b}Pooled sera were measured for neutralizing as well as binding antibodies as described in the text. Hyperimmune CVB3/0, polyclonal horse anti-CVB3 serum (ATCC); Hyperimmune Ad2, polyclonal mouse serum obtained 2 weeks after the last of four inoculations (each 3 weeks apart) with Ad2.
^{c}Neutralizing-antibody titers are expressed as the reciprocal of the highest antibody solution preventing virus-induced cytopathic effects in three of three wells.
^{d}ELISA binding antibody titers were defined as the highest serum dilution giving an absorbance value of more than 0.2 optical density unit at 405 nm above control levels.

Virus binding antibody enzyme linked immunosorbent assay. To determine the titer of anti-Ad2 binding antibody in murine sera, an enzyme-linked immunosorbent assay was constructed by coating 96-well flat-bottom plates (Dynex Technologies, Chantilly, Va.) with Ad2 that had been prepared from HeLa cell monolayers. The enzyme-linked immunosorbent assay was performed using a peroxidase detection system (mouse-hybridoma subtyping kit; Boehringer Mannheim, Indianapolis, Ind.). Briefly, 96-well plates were coated for 1 h at room temperature with 7×10³ to 8×10³TCID₅₀ of Ad2 per well. After washing and postcoating as specified by the manufacturer the plates were incubated with different dilutions of the CVB3-PL2-Ad2L1 immune murine serum (1/2, 1/20, 1/100, 1/500, 1/1,000, 1/5,000, and 1/10,000) for 30 min at 37° C. After the plates were washed, peroxidase-conjugated goat anti-mouse IgG was applied, and bound secondary antibodies were subsequently visualized using the peroxidase substrate 2,2′-azinobis(3-ethylbenzthiazolinesulfonic acid) (ABTS) provided by the kit. Color intensity was evaluated in a microplate reader (Skatron, Sterling, Va.) at a wavelength of 405 nm.

Anti-Ad2 and anti-CVB3 neutralizing-antibody assays. Neutralizing-antibody titers in murine sera were determined. Aliquots of murine sera were heated at 56° C. for 45 min prior to use. Stocks of CVB3/0 or Ad2 with known titers were diluted so that 100 to 200 infectious particles were dispensed per well of 96-well titer plates seeded the previous day with HeLa cells. Prior to dispensing, CVB3 or Ad2 was mixed with serially diluted murine sera and incubated at 37° C. for 1 h. Titers from triplicate wells were read at 48 to 96 h, when the control wells containing only virus demonstrated complete cytopathic effects (detached and rounded up cells for CVB3; rounded up and refractile cells for Ad2).

Results

Construction of the chimeric CVB3 genome and generation of progeny virus. The infectious cDNA copy of the chimeric CVB3 genome, pCVB3-PL2-Ad2L1, containing the sequence encoding the Li loop of Ad2, was constructed as described in Materials and Methods (outlined in FIG. 7). Progeny virus (CVB3-PL2-Ad2L1) was propagated on HeLa cell monolayers from supernatants of frozen and thawed, centrifugally cleared HeLa cell transfections. Cell culture supernatants containing progeny virus were cleared of cellular debris by centrifugation, subjected to titer determination on HeLa cell monolayers, and frozen in aliquots at 74° C. Viral RNA was isolated from CVB3-PL2-Ad2L1 stocks and reverse transcribed, and a fragment spanning the capsid protein 1D-2Apro junction was enzymatically amplified and sequenced to determine whether the progeny virus contained the L1 loop coding sequence. No differences were detected from the expected sequence. All chimeric virus stocks used in these experiments were derived and sequence verified in this fashion.

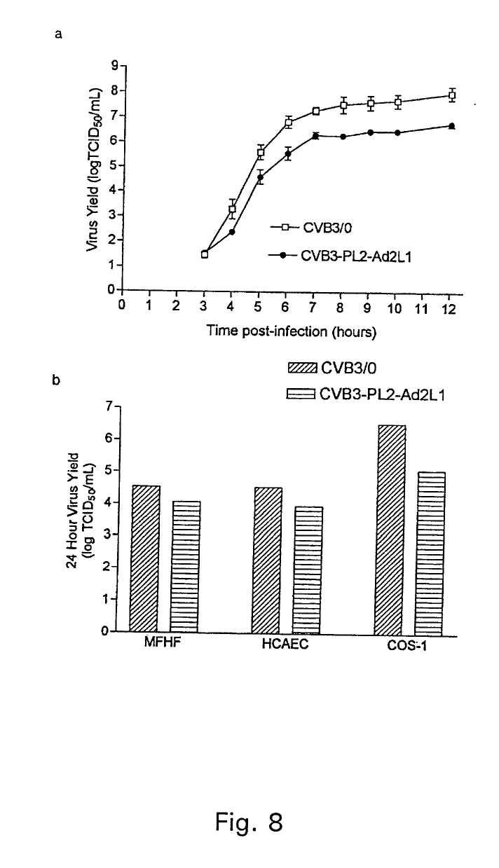

Characterization of chimeric virus replication in cell cultures. To investigate chimeric virus CVB3-PL2-Ad2L1 replication in cell culture relative to its parental strain CVB3/0, we inoculated HeLa cells, COS-1 cells, primary HCAEC cultures, and primary MFHF cultures (FIG. 8) with both viruses. The chimeric virus CVB3-PL-Ad2L1 replicated at similar rates in HeLa cell monolayers (FIG. 8a) but yielded approximately 10-fold less infectious virus titer. Under these conditions, HeLa cell cultures demonstrate advanced cytopathic effects by 7 to 9 h postinoculation. The yields of both viruses were reduced in the other cell cultures tested relative to the titers achieved in HeLa cells (FIG. 8b), with titers of the chimeric strain 0.5 to 2 log units lower than those of CVB3/0. The data suggest that expression of the Ad2 μl loop polypeptide partially attenuates virus replication relative to the parental strain.

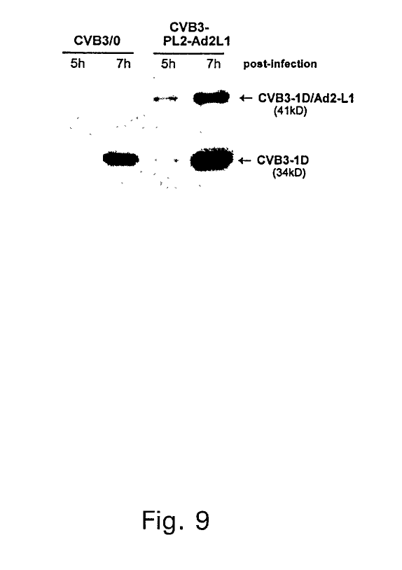

Western blot analysis of viral protein translation in infected-cell cultures. For the chimeric virus CVB3-PL2-Ad2L1 to replicate successfully, the capsid protein 1D must be cleaved by 2Apro at its carboxyl terminus, where it forms a junction with the artificially inserted Ad2 hexon L1 loop polypeptide. To investigate the efficiency of this cleavage event, we studied the processing of capsid protein 1D in infected HeLa cells by Western blot analysis. Proteins from HeLa cells inoculated either with CVB3-PL2-Ad2L1 or with CVB3/0 were separated on SDS-containing 14% polyacrylamide gels, blotted, and probed with a polyclonal horse neutralizing anti-CVB3 antibody that detects the CVB3 capsid protein 1D. Since an antibody that recognizes the Ad2 hexon L1 loop sequence on Western blots was unavailable, detection of the Ad2 polypeptide was not performed. Using the anti-CVB3 antibody, the 34-kDa CVB3 capsid protein 1D was detected at 5 h postinoculation in cells inoculated with the chimeric virus, whereas the same band was detected later, at 7 h, in the CVB3/0-inoculated cultures (FIG. 9). A prominent band in cells infected with the chimeric virus that migrated with an apparent size of a fusion protein consisting of the capsid protein 1D and the hexon L1 loop polypeptide (41 kDa) was also detected. Densitometric comparison of the Western blot signals demonstrated that CVB3-PL2-Ad2L1 overproduced both capsid protein 1D and the uncleaved 1D/Ad2 L1 loop chimeric protein by approximately 3.8-fold relative to 1D translation in CVB3/0-infected cells. These results suggested that the lower yields of chimeric virus in cell cultures might be linked to the delayed processing at the capsid protein 1D/Ad2 L1 loop junction by 2Apro.

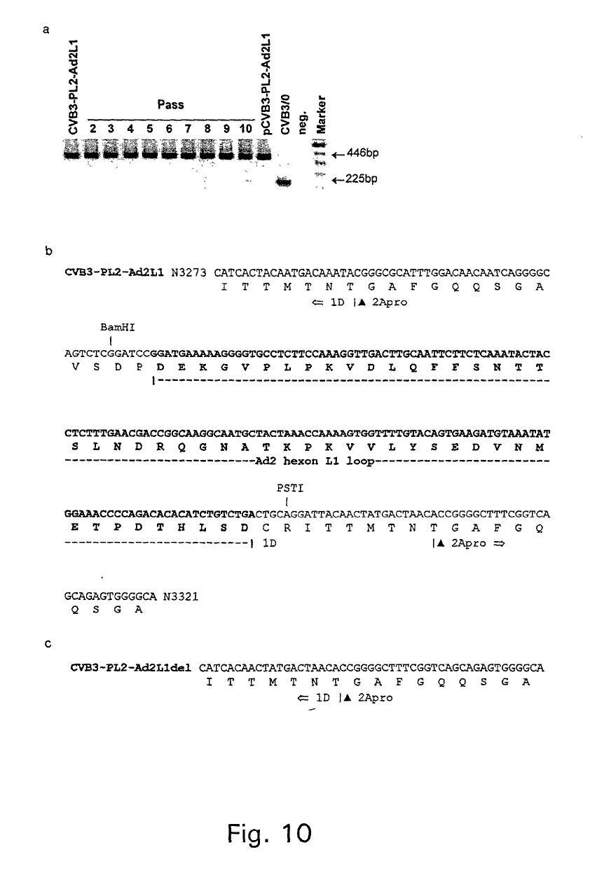

Stability of the Ad2 hexon L1 loop coding sequence in the CVB3 vector genome. Western blot data suggested that the Ad2 L1 loop coding sequence was maintained and expressed in the CVB3-PL2-Ad2L1 genome. However, an alternative hypothesis was that we were investigating a mixed population of virus, such that viral RNAs with and without the Ad2 L1 loop fragment coding sequence were being translated in the infected cells. Viral RNA with the Ad2 insert deleted might be producing the capsid protein 1D, while insert-containing RNA would be producing both 1D and the chimeric 1D-Ad2L1 loop protein. Although sequence analysis strongly suggested that the virus stocks were uniformly chimeric and not deleted with respect to the Ad2 L1 loop insert coding sequence, we tested the hypothesis by examining the CVB3-PL2-Ad2L1 RNA populations in infected HeLa cells by RT-PCR and sequence analysis. To determine the stability of the inserted sequence in the CVB3 genome as a function of time in cell culture, we concurrently passaged CVB3-PL2-Ad2L1 10 times in HeLa cells. Viral RNA was isolated from virus stocks at each pass and used as template in RT-PCRs with primers located outside of and flanking the insertion site in the CVB3 genome. Analysis of the amplimers by agarose gel electrophoresis showed that the inserted Ad2 sequence remained stable in the CBV3 genome for at least 10 passages in HeLa cell monolayers, generating the expected size of 446 bp for the insert-containing amplimer (FIG. 10a). In passages 8 and 10, we detected a low level of an amplimer that would correspond in size to that generated from a CVB3 genome with the Ad2 insert deleted. These smaller amplimers, as well as representative insert-containing bands from passages 3, 5, and 10, were isolated from agarose gels and sequenced using as sequencing primers the same primers that had been used in the RT-PCR analysis. Sequence analysis revealed that the 446-bp amplimers contained the expected Ad2 hexon Li loop coding sequence and the flanking sequences in frame with the CVB3 ORF (FIG. 10b). The smaller 225-bp fragments from pass 8 and 10 were indeed from viral genomes that had deleted the L1 loop sequence (FIG. 10c). The sequence of these deleted genomic fragments also demonstrated that the viral ORF had been maintained intact and therefore that these amplimers had not been derived from nonviable viral RNA. These results do not support the hypothesis that two different virus populations were present in the passaged CVB3-PL2-Ad2L1 or that significant deletions were generated de novo in each passage and transmitted as progeny virus. Evidence for a deleted virus population, potentially capable of replication, was obtained by amplification analysis only in two later passages. The passage data are consistent with a dominant L1 loop insert-containing viral quasispecies that remains stable through at least 10 passages in HeLa cell monolayers, suggesting that the chimeric 1D/Ad2L1 protein observed in the Western blot analysis is most probably due to a delay in 2Apro cleavage at the engineered site between 1D and the Ad2 hexon protein fragment.

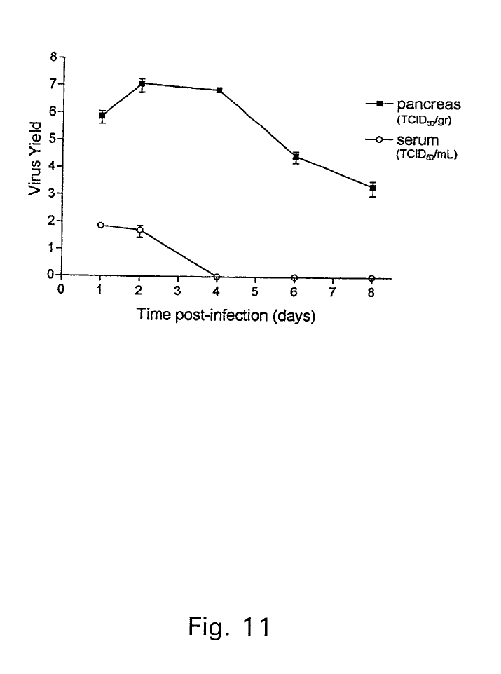

Characterization of chimeric virus replication and pathogenicity in mice. To determine if the chimeric virus replicates in mice, mice were inoculated and sacrificed on days 1, 2, 4, 6, and 8 postinoculation. Virus titers in the murine sera, pancreata, and hearts were subsequently measured on HeLa cells (FIG. 11). CVB3-PL2-Ad2L1-infected mice experienced a brief viremia with prolonged viral replication in the pancreas but without detectable virus titers in hearts.

Histopathological examinations of mice inoculated with the chimeric virus revealed healthy pancreas and heart tissues with no evidence of virus-induced lesions, in contrast to pancreatic inflammation and damage observed in CVB3/0-infected mice (data not shown). These experiments demonstrate that the chimeric virus CVB3-PL2-Ad2L1 is capable of replicating in mice and is attenuated for inducing disease in murine pancreatic tissues.

Antibody responses in mice to infection by CVB3-PL2-Ad2L1. A synthetic peptide containing the 13 amino acids of the Ad2 hexon L1 loop has been shown by to be antigenic in rabbits (Toogood et al., 1992, supra), promoting the generation of serotype-specific, anti-Ad2 neutralizing antibodies. To determine whether mice would mount an immune response against the Ad2 L1 loop polypeptide that was expressed during replication of the chimeric virus, CVB3-PL2-Ad2L1 was inoculated into BALB/c mice once, twice, or three times. Mice were sacrificed 14 days after the final inoculation. Five mice were in each group, and sera were pooled to assay for the presence of anti-CVB3 and anti-Ad2 neutralizing and binding antibodies. Antibodies in the murine sera bound immobilized Ad2 in an ELISA-based assay, ranging from 1/20 after one inoculation to 1/1,000 after three inoculations (Table 2). While anti-Ad2 neutralizing antibodies were negligible after a single inoculation, titers between ⅛ and 1/16 were obtained after three inoculations (Table 2). We also performed this experiment in C3H/HeJ mice (H-2k haplotype) with similar results, suggesting that the results were not due to a specific murine host. Ad2-binding antibodies in the sera were subtyped using an ELISA. The primary component was IgG1 at a titer of 1/1,000, with detectable IgG2a at titers between 1/20 and 1/100. No IgG2b, IgG3, or IgA were detected in the murine sera. Anti-CVB3 neutralizing antibodies were readily detected at titers ranging from 1/16 after one inoculation of CVB3-PL2-Ad2L1 to 1/64 after three exposures (Table 2). The results demonstrate that the CVB3-PL2-Ad2L1 chimeric virus induces both anti-CVB3 neutralizing antibodies and anti-Ad2 neutralizing and binding antibodies in experimentally inoculated mice and that the Ad2 hexon Li loop is antigenic in mice as well as in rabbits.

Induction of anti-Ad2 immunity in mice with preexisting anti-CVB3 immunity. CVB are common causes of human infection. Although preexisting immunity to a viral agent can protect from disease caused by the specific virus, it does not necessarily preclude reinfection by that agent as has been shown by both poliovirus (PV) vaccines and more recently developed Ad vectors. To determine whether CVB3-PL2-Ad2L1 could induce anti-Ad2 immunity in mice with preexisting immunity against the CVB3 vector, mice were inoculated once or twice (14 days apart) with CVB3/0. We have shown previously that infectious CV23/0 is cleared from mice by day 7 to 10 postinoculation. Mice were subsequently challenged with CVB3-PL2-Ad2L1 14 days after the last CVB3/0 inoculation and again 2 weeks later. Sera were isolated after sacrifice 2 weeks after the final challenge. Two weeks later, after the initial CVB3/0 inoculation, a group of five randomly chosen control mice were sacrificed. Sera from these mice were assayed for the presence of anti-CVB3 neutralizing activity; all sera expressed neutralizing anti-CVB3 antibody titers ranging between ⅛ and 1/32. Antibodies in pooled serum from mice inoculated once with CVB3/0 and then twice with CVB3-PL2-Ad2L1 were assayed by ELISA for the presence of binding antibodies. Anti-Ad2 binding antibodies from mice inoculated once with the chimeric virus were detected at titers between 1/5,000 and 1/10,000 (Table 2). Neutralizing anti-Ad2 antibodies were detected at serum dilutions between 1/16 and 1/32. These titers were between two- and fourfold higher than those observed in mice that had received only three successive inoculations of CVB3-PL2-Ad2L1. Mice that had been inoculated twice with CVB3/0 and then twice with CVB3-PL2-Ad2L1 showed binding and neutralizing antibodies detected at serum dilutions 1/10,000 and 1/32, respectively (Table 2). These data demonstrated that CVB3-PL2-Ad2L1 can induce anti-Ad2 immunity in mice with preexisting protective immunity against the CVB3 vector and that the immunity obtained was higher than that observed in mice inoculated only with the chimeric virus.

### EXAMPLE 6

### Creation of a Trivalent CVB3 Strain that Vaccinates against CVB2, CVB3 and CVB4

The BC loops of capsid protein 1D are prominent on the outside of the coxsackievirus and are also immunogenic, inducing neutralizing antibodies. This example describes the construction of a trivalent CVB3 strain that vaccinates against other CVB serotypes by expressing the BC loop regions of the other CVB types, specifically CVB2 and CVB4.

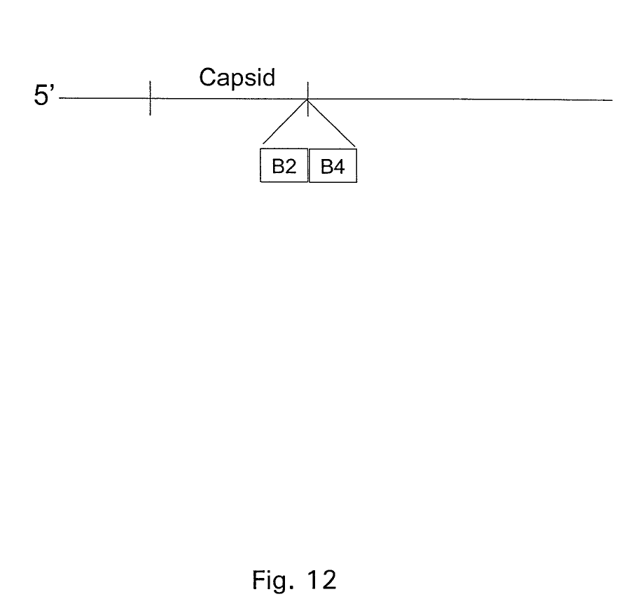

FIG. 12 diagramatically shows the CVB3 construct into which was inserted nucleic acid segments encoding the capsid protein 1D BC loops from CVB2 and CVB4. The CVB2 and CVB4 BC loop segments were inserted at the 2Apro cleavage site in a manner similar to that described for the Ad2 hexon L1 loop in Example 5.

The multi-BC loop strain was demonstrated to neutralize three of the six serotypes of CVB, as shown in Table 3 below:

TABLE 3
Neutralizing Titers from Vaccinated Mouse Sera.

| Antiserum | Virus-neutralizing titers |
|---|---|
| Anti-CVB2 | 1/8 |
| Anti-CVB4 | 1/16 |
| Anti-CVB3 | 1/128 |

### EXAMPLE 7

### Coxsackievirus-Expressed IL-4 Protects NOD Mice Against Insulin-Dependent (Type 1) Diabetes Mellitus

Insulin-dependent (type 1) diabetes mellitus (IDDM) is a disease with an incidence in the United States of about 16/1007000. IDDM is a chronic disease characterized by an autoimmune, predominantly Th1, response against the beta cells in the pancreas. The genetic background of an individual (e.g., the expression of particular MHC alleles, such as HLA-DR3) can predispose the individual to IDDM. Environmental conditions (e.g., infections, diet) are also suspected to contribute to an individual's predisposition to the disease.

Nonobese diabetic (NOD) mice are a model of human IDDM. Insulitis begins in NOD mice at about four weeks of age with glycosuria, loss of pancreatic beta cell islets, and the development of autoimmunity against several pancreatic proteins occurring by about 12 weeks. Death occurs within 3-5 weeks of the onset of these symptoms.

A variety of diverse agents, such as cytokines, rodent viruses and Freund's adjuvant, can suppress the development of diabetes in NOD mice. It has been determined that induction of a Th2 type immune response is beneficial and protective against diabetes development.

It has been shown that mIL-4 or rodent viruses can protect NOD mice from diabetes if the mice are inoculated at a young age. This example describes the suppression of IDDM in NOD mice by the administration of a CVB3-expressed murine IL-4.

Female NOD mice four weeks of age were inoculated with a CVB3 strain that expresses biologically active murine IL-4 (CVB3-PL2-mIL4/46), similar to that described in Examples 2 and 3. Controls consisted of mice inoculated with CVB3/0 or unsupplemented RPMI.

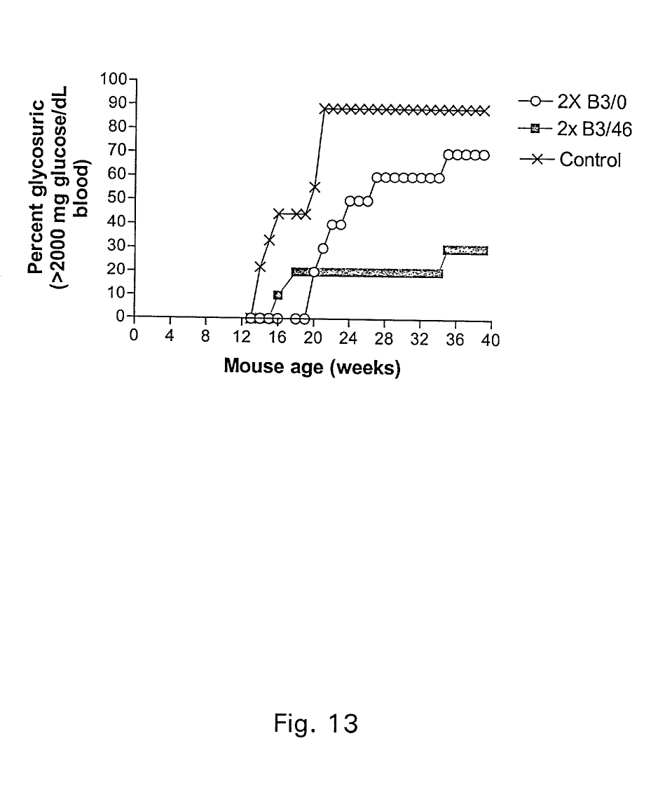

Results are shown in FIG. 13. As can be seen, CVB3-expressed murine IL-4 was able to suppress diabetes in NOD mice. Seventy percent of mice inoculated with the CVB3-PL2-mIL4/46 vector were protected from diabetes through 39 weeks of age.

### EXAMPLE 8

### Coxsackievirus Vaccination Against Insulin-Dependent (Type-1) Diabetes Mellitus (IDDM)

The etiology of IDDM suggests that coxsackie B viruses can precipitate the onset of IDDM in humans. However, the age of the person is important in this phenomenon, inasmuch as infections occurring during the first year of life appear to be key to decreased risk of IDDM, while infection in subsequent years correlates with increased risk. Studies in mice corroborate the observations made in humans, in that the earlier in life a mouse is inoculated with a rodent virus, the better is the level of protection against IDDM. This is consistent with the hygiene hypothesis that has been proposed for atopic diseases.

The data set forth in Example 7 (inoculation with CVB3/0) suggested that infection with the CVB3 alone (no IL-4 coding sequence) conferred to the mice a certain amount of protection from IDDM, as compared with the RPMI controls. The present example describes experiments designed to explore this phenomenon.

Strains of CVB3 with different virulence levels were inoculated into young NOD mice: these strains were CVB3/M (most virulent), CVB3/OL and CVB3/GA (both moderately virulent. Controls comprised inoculation with RPMI along.

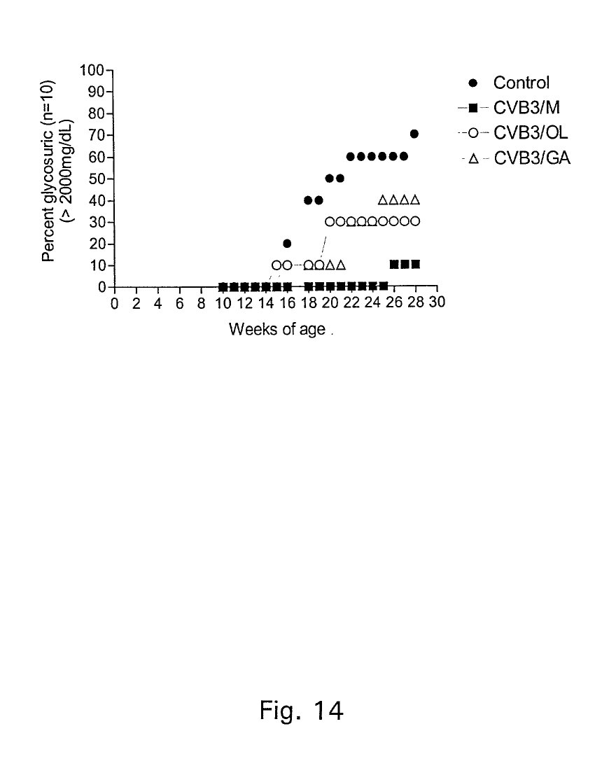

Results are shown in FIG. 14. As can be seen, inoculation of young mice with CVB3 protected 60% to 90% of the mice from the onset of IDDM. The virulent strain CVB3/M suppressed disease onset more efficiently than did the less virulent strains. Thus, CVB3 inoculation of young mice does not hasten IDDM onset; instead it generally suppresses the disease in the NOD mouse model.

The present invention is not limited to the embodiments described and exemplified above. It is capable of variation and modification within the scope of the appended claims.

We claim:

1. A vaccine for immunizing an individual against a virus, wherein the vaccine comprises:
a viral vector comprising a coxsackievirus genome modified to encode an attenuated coxsackievirus, the genome further comprising at least one cloning site for insertion of at least one expressible heterologous nucleic acid, wherein the heterologous nucleic acid encodes at least one antigenic epitope of the virus.

2. The vaccine of claim 1, wherein the virus is adenovirus and the heterologous nucleic acid encodes an Adenovirus 2 hexon loop.

3. The vaccine of claim 1, wherein the virus is human immunodeficiency virus.

4. The vaccine of claim 1, adapted to immunize an individual against a plurality of viruses.

5. The vaccine of claim 4, wherein the plurality of viruses comprise a plurality of coxsackievirus serotypes and the heterologous nucleic acid encodes a BC loop of capsid protein 1D from one or more coxsackievirus serotypes other than the viral vector serotype.

6. The vaccine of claim 1, wherein the viral vector comprises a a coxsackievirus B genome.

7. The vaccine of claim 6, wherein the coxsackievirus genome is a coxsackievirus B3 genome.

8. The vaccine of claim 7, wherein the coxsackievirus genome is modified by altering a transcription regulatory region of the genome.

9. The vaccine of claim 8, wherein the transcription regulatory region comprises a 5′ untranslated region of the genome.

10. The vaccine of claim 9, wherein the 5′ untranslated region is replaced with a 5′ untranslated region of another enterovirus genome selected from the group consisting of poliovirus and echovirus.

11. The vaccine of claim 9, wherein a uracil nucleotide at position 234 of the genome is replaced by a cytosine nucleotide or a guanine nucleotide.

12. The vaccine of claim 9, wherein a guanine nucleotide at position 233 of the genome is replaced by a cytosine nucleotide and an andenine nucleotide at position 236 of the genome is replaced by a uracil nucleotide.

13. The vaccine of claim 1, wherein the cloning site is positioned between a coding sequence for a capsid protein and a coding sequence for viral protease.

14. The vaccine of claim 1, wherein the cloning site is positioned at the start of the genome's open reading frame, and is constructed such that the inserted expressible heterologous DNA comprises a translation start codon and a 3′ sequence recognized by a viral protease.

15. A method of immunizing an individual against a virus, which comprises administering to the patient the vaccine of claim 1.

16. A composition for treating an individual for insulin-dependent diabetes mellitus, which comprises:
a viral vector comprising a coxsackievirus genome modified to encode an attenuated coxsackievirus, the genome further comprising at least one cloning site for insertion of at least one expressible heterologous nucleic acid, wherein the heterologous nucleic acid encodes a biologically active immunomodulatory protein that induces a shift from a Th1 to a Th2 immune response in the individual.

17. The composition of claim 16, wherein the heterologous nucleic acid encodes IL-4.

18. The composition of claim 16, wherein the viral vector comprises a a coxsackievirus B genome.

19. The composition of claim 18, wherein the coxsackievirus genome is a coxsackievirus 23 genome.

20. The composition of claim 19, wherein the coxsackievirus genome is modified by altering a transcription regulatory region of the genome.

21. The composition of claim 19, wherein the transcription regulatory region comprises a 5′ untranslated region of the genome.

22. The composition of claim 21, wherein the 5′ untranslated region is replaced with a 5′ untranslated region of another enterovirus genome selected from the group consisting of poliovirus and echovirus.

21. The composition of claim 19, wherein a uracil nucleotide at position 234 of the genome is replaced by a cytosine nucleotide or a guanine nucleotide.

22. The composition of claim 19, wherein a guanine nucleotide at position 233 of the genome is replaced by a cytosine nucleotide and an andenine nucleotide at position 236 of the genome is replaced by a uracil nucleotide.

23. The composition of claim 16, wherein the cloning site is positioned between a coding sequence for a capsid protein and a coding sequence for viral protease.

24. The composition of claim 16, wherein the cloning site is positioned at the start of the genome's open reading frame, and is constructed such that the inserted expressible heterologous DNA comprises a translation start codon and a 3′ sequence recognized by a viral protease.

25. A method of treating, preventing or suppressing onset of insulin-dependent diabetes mellitus in an individual, which comprises administering to the individual the composition of claim 16.

26. A method of suppressing onset of insulin-dependent diabetes mellitus in an individual, which comprises inoculating the individual as a juvenile or infant with a coxsackievirus.

27. The method of claim 26, wherein the coxsackievirus is a coxsackie 2 virus.

28. The method of claim 27, wherein the coxsackievirus is CVB3.

29. The method of claim 28, wherein the coxsackievirus is a virulent strain of CVB3.


SEQUENCE LISTING (28 sequences)

SEQ ID NO: 1 - DNA, 85 bp, Coxsackievirus
atcactacaatgacaaatacgggcgcatttggacaacaatcaaggggcagcgtatgtggggaactacagggtaatgggtctcaac

SEQ ID NO: 2 - DNA, 80 bp, Coxsackievirus
tactcgatcactacaatgacaaatacgggcgcatttggacaacaatcaggggcagcgtatgtggggaactacagggtagt

SEQ ID NO: 3 - protein, 34 aa, Coxsackievirus
SGVTTTRQSITTMTNTGAFGQQSGAVTLEMPGSA

SEQ ID NO: 4 - protein, 25 aa, Coxsackievirus
MKSNSITTMTNTGAFGQQSGAVYVG

SEQ ID NO: 5 - DNA, 27 bp, Coxsackievirus
atgggaaattcgagctcgatgcctggc

SEQ ID NO: 6 - DNA, 28 bp, Coxsackievirus
atgaaaagcgcatgcgggttttcaaggt

SEQ ID NO: 7 - DNA, 74 bp, Coxsackievirus
actactaggcaaagcatcactacaatgacaaatacgggcgcatttggacaacaatcagggcagtctcggatcca

SEQ ID NO: 8 - DNA, 62 bp, Coxsackievirus
gaattctgcagatcaattaccaccatgaccaacacggggcgcatttggacaatcaggggcag

SEQ ID NO: 9 - protein, 5 aa, Coxsackievirus
NTGAF

SEQ ID NO: 10 - protein, 11 aa, Coxsackievirus
YRVMGLNYSIT

SEQ ID NO: 11 - protein, 58 aa, Coxsackievirus
SGVTTTRQSITTMTNTGAFGQQSGAVTLEDPRVPSSNSITTMTNTGAFGQQSGAVYVG

SEQ ID NO: 12 - protein, 59 aa, Coxsackievirus
SGVTTTRQSITTMTNTGAFGQQSGAVTLEMPGSAMKSNSITTMTNTGAFGQQSGAVYVG

SEQ ID NO: 13 - DNA, 69 bp, Coxsackievirus
atgggaaattcgagctccgtacccggggatcctctagagtcgacctgcaggcatgcgggttttcaagga

SEQ ID NO: 14 - DNA, 55 bp, Coxsackievirus
atgggaaattcgagctcgatgcctggcatgaaaagcgcatgcgggttttcaaggt

SEQ ID NO: 15 - protein, 23 aa, Coxsackievirus
MGNSSSVPGDPLESTCRHAGFQG

SEQ ID NO: 16 - protein, 18 aa, Coxsackievirus
MGNSSSMPGMKSHAGFQG

SEQ ID NO: 17 - DNA, 138 bp, Coxsackievirus
actactaggcaaagcatcactacaatgacaaatacgggcgcatttggacaacaatcaggggcagtctcggatccagaattctgcagatcaattaccaccatgaccaacaccggggcgcatttggacaatcaggggcag

SEQ ID NO: 18 - protein, 46 aa, Coxsackievirus
TTRQSITTMTNTGAFGQQSGAVSDPEFCRCITTMTNTGAFGQSGAV

SEQ ID NO: 19 - DNA, 124 bp, Coxsackievirus
catcactacaatgacaaatacgggcgcatttggacaacaatcaggggcagtctcggatcctaggatatcctgcaggattacaactatgactaacaccggggctttcggtcagcagagtggggca

SEQ ID NO: 20 - protein, 41 aa, Coxsackievirus
ITTMTNTGAFGQQSGAVSDPRISCRITTMTNTGAFGQQSGA

SEQ ID NO: 21 - DNA, 268 bp, Coxsackievirus
catcactacaatgacaaatacgggcgcatttggacaacaatcaggggcagtctcggatccggatgaaaaaggggtgcctcttccaaaggttgacttgcaattcttctcaaatactacctctttgaacgaccggcaaggcaatgctactaaaccaaaagtggttttgtacagtgaagatgtaaatatggaaaccccagacacacatctgtctgactgcaggattacaactatgactaacaccggggctttcggtcagcagagtggggca

SEQ ID NO: 22 - protein, 89 aa, Coxsackievirus
ITTMTNTGAFGQQSGAVSDPDEKGVPLPKVDLQFFSNTTSLNDRQGNATKPKVVLYSEDVNMETPDTHLSDCRITTMTNTGAFGQQSGA

SEQ ID NO: 23 - DNA, 31 bp, Coxsackievirus
tccggatgaaaaaggggtgcctcttccaaag

SEQ ID NO: 24 - DNA, 30 bp, Coxsackievirus
gcctctgcagtcagacagatgtgtgtctgg

SEQ ID NO: 25 - DNA, 20 bp, Coxsackievirus
ctagactctgccaatacgag

SEQ ID NO: 26 - DNA, 21 bp, Coxsackievirus
gtgctcactaagaggtctctg

SEQ ID NO: 27 - DNA, 49 bp, Coxsackievirus
catcacaactatgactaacaccggggctttcggtcagcagagtggggca

SEQ ID NO: 28 - protein, 16 aa, Coxsackievirus
ITTMTNTGAFGQQSGA